(19) United States
(12) Patent Application Publication
(10) Pub. No.: US 2010/0191215 A1
(43) Pub. Date: Jul. 29, 2010

(54) PRESSURE PULSE ACTUATING DEVICE FOR DELIVERY SYSTEMS

(75) Inventors: Oren Globerman, Herzlia Pituach (IL); Mordechay Beyar, Herzlia Pituach (IL)

Correspondence Address: MARTIN D. MOYNIHAN d/b/a PRTSI, INC., P.O. BOX 16446, ARLINGTON, VA, 22215, US

(73) Assignee: By-pass ,Inc., Orangeburg, NY (US)

(21) Appl. No.: 12/663,859

(22) PCT Filed: Jun. 12, 2008
(86) PCT No.: PCT/IL2008/000802

§ 371 Date: Dec. 10, 2009

Related U.S. Application Data
(60) Provisional application No. 60/943,311, filed on Jun. 12, 2007.

(51) Int. Cl. A61M 5/30; B23P 11/00

(52) U.S. Cl. 604/500; 604/68; 29/428

(57) ABSTRACT

A system and method for delivering medicament to tissue, using a pulse pressure source and optional pre-pressuring. Optionally, the delivery system is modular and/or adjustable for various applications. Optionally, the treated tissue is a blood vessel wall. Optionally, the delivery system is mechanical.

### RELATED APPLICATIONS

This application claims the benefit under 119(e) of 60/943,311, filed Jun. 12, 2007, by, inter alia, Mordechay Beyar, the disclosure of which is incorporated herein by reference.

The present application also relates to international patent applications PCT/IL2005/000749 and PCT/IL2006/000087 and U.S. patent application Ser. Nos. 11/335,317 and 11/609,451, an inventor common to all of these applications is Mordechay Beyar, the disclosures of which are incorporated herein by reference.

### FIELD OF THE INVENTION

The present invention relates to the delivery of materials, for example, high speed needle-less injections.

### BACKGROUND OF THE INVENTION

Balloon catheters which incorporate drug delivery capabilities were suggested for use in treatments of blocked bodily vessels, such as Percutaneous Transluminal Coronary Angioplasty (PTCA) procedures, especially where prevention of restenosis is needed.

U.S. Pat. Nos. 5,614,502 and 6,716,190, the disclosures of which are incorporated herein by reference, describe methods of material delivery inside the body, including transvascularly.

W. J. Walker, I. M. Faireley “A simplified technique for the per catheter delivery of Isobutyl 2—Cyanoacrylate in the Embolisation of Bleeding Vessels”, Journal of Interventional Radiology 1987 2, 59-63, the disclosure of which is incorporated herein by reference, describes the injection of glue into a lumen and against walls of an artery, in order to block it.

U.S. Pat. Nos. 5,611,775, 5,087,244, 4,994,033, 5,232,444 and 6,280,414, the disclosures of which are incorporated herein by reference, describe systems for delivering a drug to the wall of a blood vessel.

Pumps or syringes are used to inflate the balloon and/or to press the drug through the catheter lumen(s) and into the treated tissue, generally at a pressure which does not exceed 20 atmospheres. An example of such a device is the Indeflator® syringe of Advanced Cardiovascular Systems, Inc., Santa-Clara, Calif., USA as described in U.S. Pat. No. 5,611,775, the disclosure of which is incorporated herein by reference. A similar syringe type is used both for inflating an internal balloon for opening the blocked vessel, and for injecting the drug.

Pressurizing pulse systems are used as actuators in needle-less injectors for administrating medication to a percutaneous or subcutaneous skin tissue zones. U.S. Pat. Nos. 5,599,302, 5,919,159 and 5,891,085, the disclosure of which are incorporated herein by reference, present a needle-less injecting device that uses a self-contained gas-spring as a device actuating mechanism. A benefit of the gas-spring, as mentioned in these patents, is that it has low volume, and provides a substantially uniform force along its travel.

### SUMMARY OF THE INVENTION

A broad aspect of some embodiments of the present invention relates to a high pressure pulse gun used for actuating medicament injection into a live tissue. Said pressure pulse gun may be attached to a fluid dispensing system (e.g. a drug delivery catheter), filled before, during or after said attaching with a medicament or other fluid material, in order to rapidly inject it by releasing a high pressure pulse. In an exemplary embodiment of the invention, said delivery system is especially designed for injecting a medicament as a plurality of fine jets capable of piercing and/or penetrating a live tissue.

In an exemplary embodiment of the invention, said pulse gun is set to provide pulse/s that exceeds 50 atmospheres, 100 atmospheres, 200 atmospheres, 400 atmospheres, 600 atmospheres, or higher or lower or intermediate pressures. Said pressure pulse gun may be disposable or intended for repeated use, while optionally includes disposable parts, such as heads.

In an exemplary embodiment of the invention, said pulse gun, in a single form and/or as different implementations, is used for a plurality of different drug delivery applications, including but not limited to: in vivo or ex vivo drug deliveries, coronary applications, oncological applications, atrial fibrillation treatments, digestive tract treatments, urinary tract treatments, treatment of tumors in bodily organs and vessels, intratumor chemotherapy, dermatologic and/or cosmetic applications, transmucosal applications, buccal cavity treatments and/or gene therapy. In an exemplary embodiment of the invention, one or more adjustments can be applied to a particular instance of a pulse gun to switch it from one exemplary application to a second, optionally said adjustment(s) can be performed by medical personnel prior and/or during treatment(s). In an exemplary embodiment of the invention, said adjustments include one or more of changing the applied force of pulse gun, for example by changing an inner gas pressure of gas spring or for example by changing compression of a coil spring. In an exemplary embodiment of the invention, said adjustment include switching to a different drug delivery system and/or a connecting element and/or an adapter or to any other connectable element (e.g. a tip) having different design and/or different applicability and/or different contents.

In an exemplary embodiment of the invention, said pulse gun is automatic or semi-automatic, e.g., it can self re-cock thus eliminating the need of manual re-cocking by the operator between two adjacent shots.

In an exemplary embodiment of the invention, the pressure pulse gun includes a high energy source, that when initiated, can produce high pressure pulse(s) as described above. Optionally, said high energy source is a gas-spring, i.e. a type of spring that uses a compressed gas, contained in a cylinder and variably compressed by a piston, to exert a force. Optionally, said gas-spring is a commercially available (for example by DADCO, Plymouth, Mich., USA or by Hyson, Brecksville, Ohio, USA), and is discrete and self-contained for replaceability. Optionally or alternatively, said pulse gun may include other energy source types, such as a disk spring and/or a coil spring and/or a powered vacuum-compression unit and/or a gas propellant unit and/or a gas generating pyrotechnic charge and/or pneumatic or hydraulic types of energy sources, such as a hydraulic accumulator, or any combination of the above. Optionally, said high-energy source is a gas cylinder containing condensed gas, such as CO2 or N2, that may be in a direct communication with a piston (e.g. a “floating piston”), optionally in a selective manner by the operator. Optionally, if the pressure pulse gun is an automatic or semi-automatic type, certain mechanisms may be applied in order to pull back said piston to a desired direction after a single shot is made, as a self-re-cocking mechanism (“blow-back action”).

In an exemplary embodiment of the invention, the pressure pulse gun includes a trigger mechanism for initiating a stored energy release, optionally energy stored in a pre-compressed spring (e.g. a gas spring), in order to produce force. Optionally, said trigger is operated manually.

In an exemplary embodiment of the invention, the pressure pulse gun includes a safety mechanism that may be used to prevent any mishandling and/or unintentional operation of the pressure pulse gun. Optionally, said safety mechanism includes a trigger safety lock, which may selectively enable or disable trigger/gun operation depending on lock's position, and/or a magazine safety element, which may selectively enable or disable trigger operation depending on whether or not a drug delivery system (e.g., “a magazine”) is safely coupled to the pressure pulse gun outlet.

In an exemplary embodiment of the invention, the pressure pulse gun includes a pusher, which is used to deliver a substantially linear force that was released by a high energy source, onto a coupled drug delivery system. Optionally, the pusher is operatively connected or is adjacent to the high energy source (e.g. to a free end of the gas spring piston).

In an exemplary embodiment of the invention, the pressure pulse gun includes a plunger, which is used to press an adequate volume of the medicament from a medicament reservoir into the drug delivery system. Optionally, said plunger is operatively connected or is adjacent to a pusher. In an exemplary embodiment of the invention, the volume of medicament expelled is 0.05 cc, 0.1 cc, 0.2 cc, 0.4 cc, 1 cc, 3 cc, 5 cc, or higher or lower or intermediate value. In an exemplary embodiment of the invention, the pressure pulse gun further includes at least one initial-pressure pump. Optionally, said pump(s) is intended to produce initial higher pressure within a drug delivery system (e.g. in order to inflate a PTCA balloon) and/or an initial higher pressure in a medicament reservoir. Optionally, said pump(s) is a commercially available PTCA pump/syringe and/or with regular working pressures between 4-10 atmospheres and a maximum pressure of approximately 15 atmospheres, approximately 30 atmospheres, approximately 50 atmospheres, or higher or lower or intermediate value. Optionally, the pressure pulse gun is sealed, optionally at a factory, under said initial higher pressure so no medicament may be dispensed out of it, and is opened for fluid delivery once the high pressure pulse is released.

In an exemplary embodiment of the invention, the medicament delivery system includes a lumen and a perforated distal end. Optionally, said medicament delivery system is a balloon and/or a drug delivery catheter intended for treating live tissues in vivo, wherein the perforated distal end may be an at least partially perforated membrane or balloon. Alternatively, said medicament delivery system is a cap element intended for treating live exterior tissue, said cap element being at least partially perforated. Optionally, said cap element is a tip.

Some embodiments of the present invention include a method for pressurizing a medicament into a live tissue, the method comprising:

(a) providing a pressure pulse gun having a high energy source that is capable of producing at least one pulse equal or higher than 50 or 100 atmospheres;

(b) optionally coupling said pressure pulse gun to a fluid channel that includes a perforated distal end; said fluid channel is capable of containing a fluid until the pressure pulse gun is activated;

(c) optionally locating said distal end of said fluid channel on or adjacent to a live tissue section to be treated; and

(d) initiating the high energy source, so that the pressure pulse gun is activated to produce at least one pulse wave equal or higher than 50 or 100 atmospheres into the fluid channel; wherein said pulse wave promotes a rapid injection of a pre-filled fluid through the perforated distal end into the preferred live tissue; wherein said fluid is infiltrated into said live tissue as separated piercing jets.

An aspect of some embodiments of the invention relates to an injector configured to provide both a pre-pressurization of a medicament and a pressure pulse to a medicament. In an exemplary embodiment of the invention, a single medicament injector includes two pistons, one which is advanced to provide pre-pulse pressure and one which is advanced by a pressure pulse. Optionally, the two pistons are concentric. Optionally, the pistons define storage locations for medicament components.

In an exemplary embodiment of the invention, the total amount of medicament is between 0.1 and 5 cc, optionally 2 cc. In an exemplary embodiment of the invention, a two component storage is used. Optionally, a first component is a medicament solution (including an active agent), 0.01-0.5 cc, optionally 0.04 cc (in case of rapamycin), optionally 0.25 cc in case of taxol. A second component is optionally saline solution. Exemplary ratios between the component volumes are: between 1:1 to 1:200, for example, 1:5-1:100, optionally 1:49 (rapamycin), optionally 1:7 (taxol).

An aspect of some embodiments of the invention relates to reducing pressure loss during a pressure pulse application by reducing loss to pressure measurement means and/or source. Optionally, the pressure loss is reduced by providing a valve, for example, a one way valve or a sealable valve, optionally automatically sealed by electronic means. Optionally or alternatively, pressure loss is reduced by providing a narrowed fluid channel which will significantly retard a pressure pulse.

An aspect of some embodiments of the invention relates to a safety system which reports and/or allows pressure pulse application only if pre-pressure is applied. In an exemplary embodiment of the invention, a mechanical means senses a deformation of a chamber associated with pressurizing medicament. Optionally or alternatively, an electronic or electrical sensor is used.

An aspect of some embodiments of the invention relates to a flexible pulse gun kit available with a range of attachments, including, for example, one or more of multiple tips, multiple application conveyers (e.g., flexible or rigid tubes), multiple drug dosages and/or multiple drug types. In an exemplary embodiment of the invention, the gun is adjustable to provide a desired pressure pulse size and/or volume. Optionally or alternatively, instructions matching up various needs and dosages and/or gun settings are provided as part of a kit.

An aspect of some embodiments of the invention relates to a multi-part assembler including separable gun, medicament reservoir and injector and catheter adapter. In an exemplary embodiment of the invention, the pulse gun is attached to a catheter only after the catheter is advanced to a treatment region and optionally pre-pressurized. Alternatively, pre-pressurization is provided by a motor on the gun that advanced a piston on the injector.

In an exemplary embodiment of the invention, a pulse gun includes a spring which generates pressures and/or injection volumes that are higher than common in the art. For example, an injection pulse is between 12 and 30 bar, optionally higher, optionally with stronger outer and/or inner balloons. Exemplary injection volumes are between 0.005-0.1 cc up to 0.01-0.03 cc (for coronary vessels), from 0.01-0.15 cc up to 0.06-0.08 cc (for peripheral vessels). Higher or lower amounts may be used as well.

An aspect of some embodiments of the invention relates to a multi-part assembler including separable gun, medicament reservoir and injector and catheter adapter.

An aspect of some embodiments of the invention relates to a method of releasing air from a balloon system in which a flexible tube is provided at the end of a system to be drain and wherein said tube is sealed during operation of the balloon system by pressure in a surrounding balloon. In an exemplary embodiment of the invention, air drainage is not through patent holes, to prevent sealing of the holes by medicament residue. Optionally, the drainage tube has a larger cross-sectional area and/or otherwise reduced resistance to air flow than the holes, so that drainage will preferentially be through the tube. For example, the cross-sectional area may be, for example, greater by a factor of 2, 3, 4 or more, or intermediate factors.

An aspect of some embodiments of the invention relate to a connector which couples a plurality of pressure sources to a single treatment element, for example, a discharge tip or a balloon. Optionally, three pressure sources are coupled, with at least one being a pressure source.

An aspect of some embodiments of the invention relates to a method of shaping a pressure pulse in which an end of the pulse is cut off by a pressure application device, in addition to or instead of such cutoff by a valve at an application point. In an exemplary embodiment of the invention, the pressure cut off is provided by configuring a pressure chamber so that when a pulse is completed, the chamber is depressurized. In an exemplary embodiment of the invention, the depressurization uses the same tool as used for pressurization. For example, a piston used for pressurizing, once it sufficiently advances, defines an opening out of the chamber, thereby reducing pressure. As noted, such pulse forming can be used to augment pulse forming provided by a valve, such a balloon valve, which only operates above a certain pressure threshold.

There is provided in accordance with an exemplary embodiment of the invention, a medicament injector, comprising:

(a) a hollow body having an outlet;

(b) a first piston configured to increase a pressure inside said hollow body; and

(c) a second piston configured to increase transmit a pressure pulse into said hollow body.

In an exemplary embodiment of the invention, said second piston travels within said first piston. Optionally or alternatively, the injector includes a manual handle for advancing said first piston.

In an exemplary embodiment of the invention, the injector includes a pressure release pathway defined between said pistons and coupled to said hollow of said hollow body during only part of a relative position of said two pistons.

In an exemplary embodiment of the invention, the injector is prefilled with at least one medicament component. Optionally, the injector is prefilled with at least two medicament components, each in a separate chamber of said injector.

In an exemplary embodiment of the invention, the injector includes a rotational mechanism which advances said first piston.

In an exemplary embodiment of the invention, a medicament delivery system comprising an injector as described above, a pressure pulse source coupled to said second piston and a medicament delivery tip coupled to said outlet, is provided.

There is provided in accordance with an exemplary embodiment of the invention, a method of delivering a medicament, comprising:

(a) pre-pressuring a delivery tube using a medicament injector; and

(b) applying a pressure pulse to said delivery tube using said injector to pass on a pulse.

In an exemplary embodiment of the invention, the method comprises attaching a pulse source to said injector after navigating said delivery tube to a treatment area.

There is provided in accordance with an exemplary embodiment of the invention, a method of assembling a medicament delivery system, comprising:

providing a pulse gun;

selecting from a plurality of application tubes, suitable for different tissue types, an application tube;

attaching said application tube to said gun.

In an exemplary embodiment of the invention, the method comprises selecting a medicament source from a plurality of available sources.

In an exemplary embodiment of the invention, the method comprises adjusting a pulse setting of said system according to said selection.

There is provided in accordance with an exemplary embodiment of the invention, a medicament injector, comprising:

(a) a first medicament chamber;

(b) a second medicament chamber;

(c) an activatable separator between said chambers; and

(d) a pressure source and an outlet, both couplable to said chambers.

In an exemplary embodiment of the invention, said pressure source comprises at least one piston which opens said separator when advanced.

There is provided in accordance with an exemplary embodiment of the invention, a pulse gun assembly, comprising separable components including at least:

(a) a delivery tip;

(b) a medicament injector; and

(c) a pulse gun.

In an exemplary embodiment of the invention, the method comprises a catheter adapter adapted to couple said delivery tip to said injector and to a second pressure source.

There is provided in accordance with an exemplary embodiment of the invention, a medicament delivery balloon valve, comprising:

(a) a first, inner balloon;

(b) a second, outer balloon with at least one medicament delivery pore provided therebetween; and

(c) at least one drainage channel which couples a space between said balloons to outside of said valve, said channel configured to be collapsed by inflating said inner balloon.

In an exemplary embodiment of the invention, said drainage tube has a cross-sectional area at least as large as all of said at least one pore.

There is provided in accordance with an exemplary embodiment of the invention, a medicament injector, comprising:

(a) body defining a medicament chamber;

(b) a piston adapted to convey a pressure pulse to said chamber; and

(c) a pressure release passageway which selectively couples to said medicament chamber by pulse providing motion of said piston, after a movement of said piston and a conveyance of a pressure pulse to said chamber.

There is provided in accordance with an exemplary embodiment of the invention, an actuator for fluid injection into a human tissue, wherein the actuator is capable of producing a pressure pulse wave with a magnitude higher than 50 atmospheres, said injection is in the form of a plurality of jet streams capable of piercing into said human tissue.

In an exemplary embodiment of the invention, the actuator comprises a delivery tip adapted to generate said streams and adapted for skin treatment.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1I illustrate an exemplary drug delivery system for urological treatment, in accordance with an exemplary embodiment of the invention;

FIG. 2 presents a flowchart of a method of utilizing a drug delivery system that includes a pressure pulse gun, in accordance with an exemplary embodiment of the invention;

FIGS. 3A-3D illustrate an exemplary drug delivery system for intra-nasal treatment, in accordance with an exemplary embodiment of the invention;

FIGS. 4A-4B illustrate an exemplary perforated tip for exterior tissue treatment, in accordance with exemplary embodiments of the invention;

FIGS. 5A-5C are a schematic illustration of a complete drug delivery system for in-vivo treatment that incorporates a pressure pulse gun, medicament and PTCA pumps and pressure sensitive valves, in accordance with an exemplary embodiment of the invention;

FIGS. 5D-5E illustrate an exemplary drug delivery system for peripheral blood vessel treatment, in accordance with an exemplary embodiment of the invention;

FIGS. 5F-5G illustrate isometric and cut views of an exemplary medicament pump, in accordance with an exemplary embodiment of the invention;

FIGS. 5H-5I illustrate isometric views of an exemplary pressure pulse gun for in-vivo blood vessel treatments, in accordance with an exemplary embodiment of the invention;

FIG. 6 presents a flowchart of a method of utilizing an in-vivo drug delivery system as schematically illustrated in FIGS. 5A-5C, in accordance with an exemplary embodiment of the invention;

FIG. 7A-7C present cross section views of the exemplary drug delivery system illustrated in FIGS. 5D-5E, in accordance with exemplary embodiments of the invention;

FIGS. 8A-8C present several operational modes of a pressure pulse gun actuating mechanism, in accordance with an exemplary embodiment of the invention;

FIGS. 9A-9C illustrate an exemplary drug delivery system for coronary blood vessel treatments, in accordance with an exemplary embodiment of the invention;

FIGS. 10A-10C illustrate a pre-pulse and pulse medicament chamber, in accordance with an exemplary embodiment of the invention;

FIGS. 11A-11C illustrate a catheter coupling element in accordance with an exemplary embodiment of the invention;

FIGS. 12A-12B illustrate a pulse delivery device, in accordance with an exemplary embodiment of the invention;

FIGS. 13A and 13B are exploded views of an assembled drug delivery device, in accordance with an exemplary embodiment of the invention;

FIGS. 14A-14D illustrate an assembled drug delivery device, in accordance with an exemplary embodiment of the invention;

FIG. 15A and FIG. 15B each illustrates a mixing injection system, in accordance with exemplary embodiments of the invention;

FIGS. 16A-16D illustrate an alternative injector design with optional pressure release, in accordance with an exemplary embodiment of the invention; and

FIG. 17 show an alternative catheter adaptor for peripheral vessel use, in accordance with an exemplary embodiment of the invention; and

FIGS. 18A and 18B illustrate a balloon valve draining mechanism, in accordance with an exemplary embodiment of the invention.

### DETAILED DESCRIPTION OF EXEMPLARY EMBODIMENTS

### Overview

Various examples of pulse-medicament delivery systems are described herein. Generally, such a system includes a delivery tip, an optional valve for shaping the delivery, a medicament reservoir and a pulse source. In some embodiments of the invention, pre-pressurizing is provided. In general, embodiments with longer delivery pathways may utilize pulse shaping means, such as pre-pressurizing, valves at the delivery tip and/or valves to prevent pressure loss.

Before explaining at least one embodiment of the invention in detail, it is to be understood that the invention is not necessarily limited in its application to the details of construction and the arrangement of the components and/or methods set forth in the following description. The invention is capable of other embodiments or of being practiced or carried out in various ways.

### Example 1

### Urological Vessels Treatments

Some exemplary uses of drug delivery for urological treatments are described in International patent application number PCT/IL2006/000087 to Beyar et al, the disclosure of which is fully incorporated herein by reference.

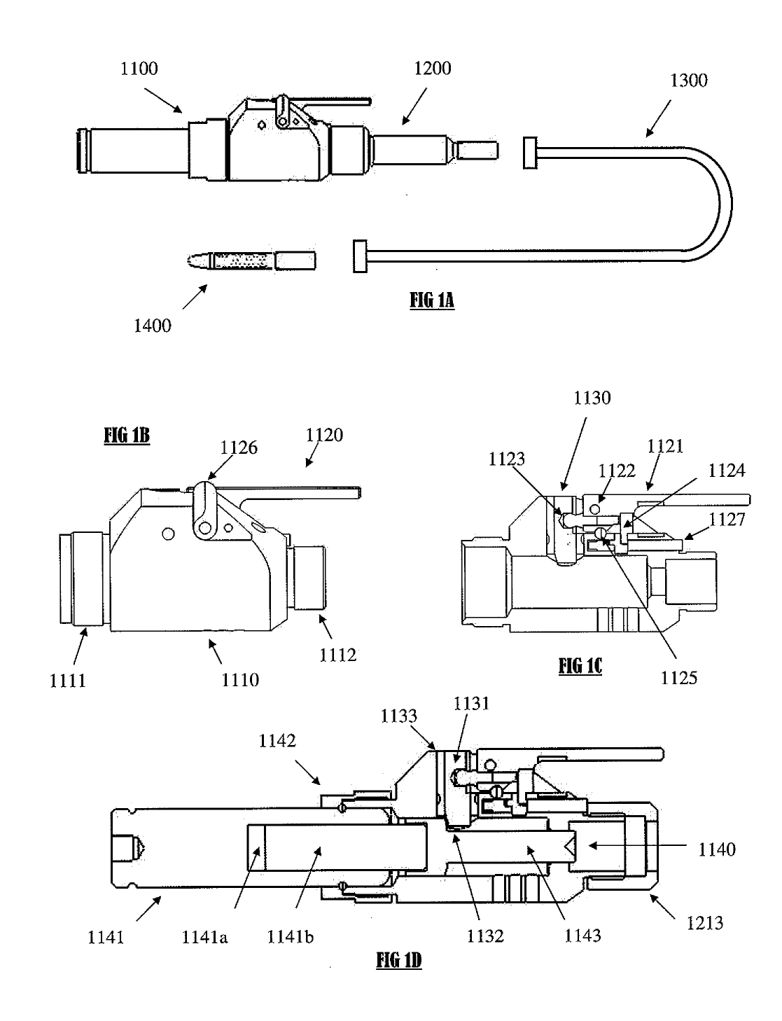

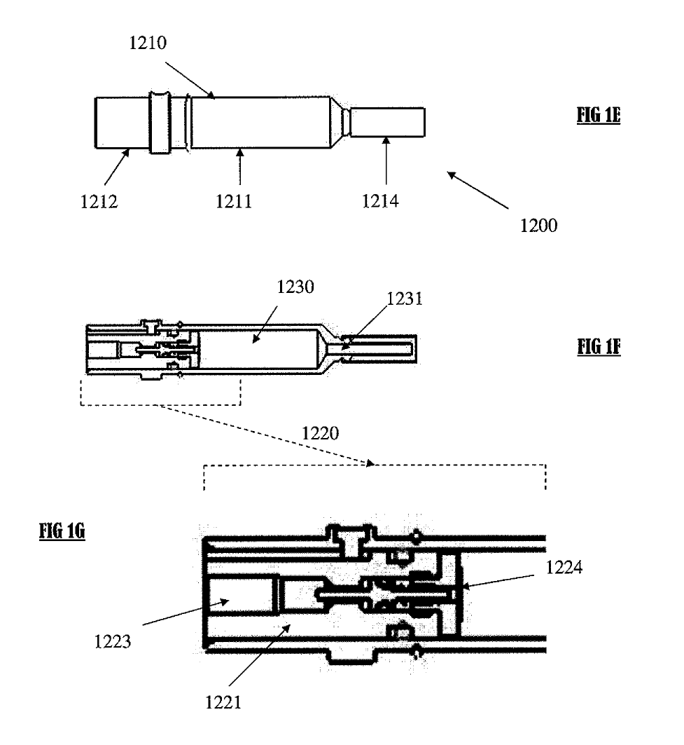

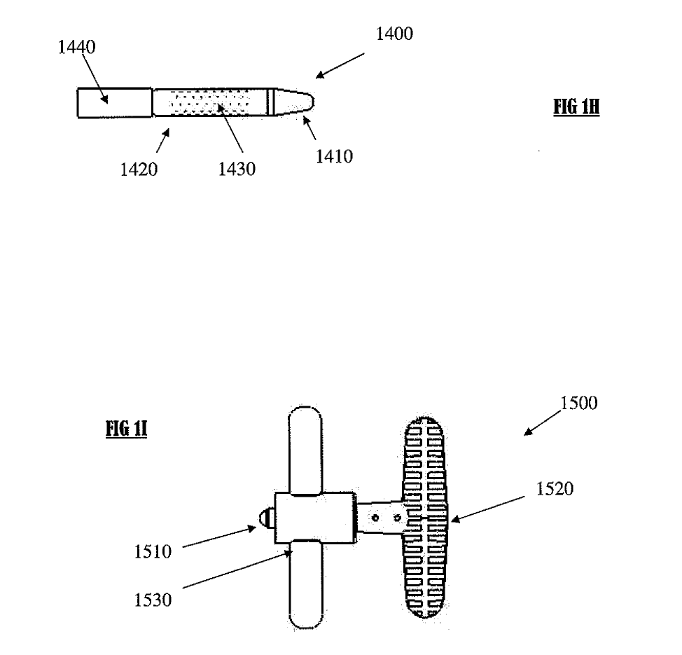

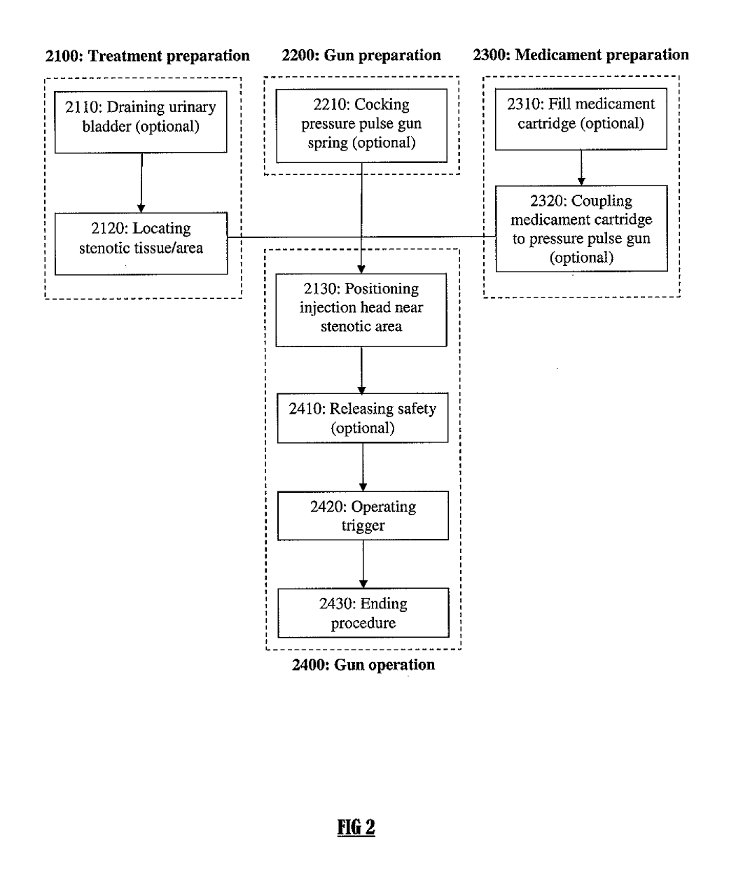

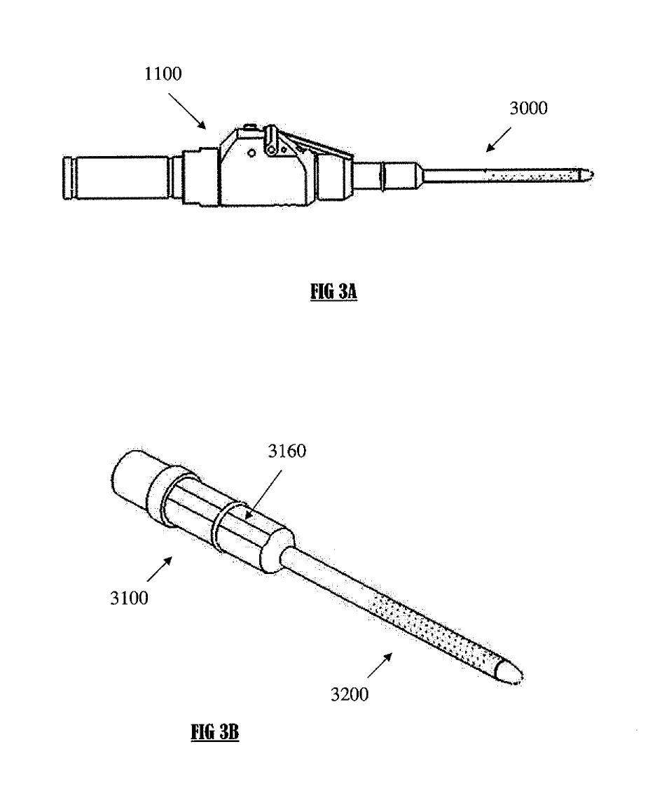

FIGS. 1A-1I, present an exemplary system and FIG. 2 presents a method, for performing drug delivery treatment for treating bodily organs or vessels. Optionally, said treatment is for treating stenotic urological vessels, such as the urethra and/or for treating the prostate. FIG. 1A shows an exemplary urological drug delivery system separated to its main parts: a pressure pulse gun 1100, a medicament cartridge 1200, a hose 1300 and an injection head 1400.

FIGS. 1B-1D describe exemplary pressure pulse gun 1100. Gun 1100 include a housing 1110, a trigger assembly 1120, a stopper assembly 1130 and an inner mechanism 1140. Trigger assembly 1120 is optionally used to initiate a sequence of events that eventually produces a force needed for the creation of a predetermined pressure pulse. Stopper assembly 1130 is optionally used to withhold actuation of inner mechanism 1140, until trigger assembly 1120, to which it is engaged, is activated Inner mechanism 1140 optionally contains the stored energy (mechanically and/or chemically and/or physically etc.) that may produce the desired force, when released.

In an exemplary embodiment of the invention, and as shown in FIG. 1D, inner mechanism 1140 incorporates a spring 1141, a spring connecting nut 1142 and a pusher 1143. Optionally, spring 1141 is a gas spring having a pressure chamber 1141a, defined by a tubular sleeve and a pair of sealed ends, and a rod 1141b. In an exemplary embodiment of the invention, gas spring 1141 can be filled with gas until a desired inner pressure is met, which is calculated according preferred work forces during spring release. Optionally, gas spring 1141 is of a commercially available design. In an exemplary embodiment of the invention, gas spring 1141 has work pressures which exceed 300N, 1000N, 3000N, and 5,000N, or have higher or lower or intermediate values. Exemplary commercial available gas springs that may be adequate for producing such forces can be DADCO Ultra Force™ Nitrogen Gas Springs types U.0175 (with work forces of approximately 1,700N-2,650N) or U.0325 (with work forces of approximately 3,200N-5,150N). Optionally, the maximal work pressures may exceed much higher values, such as optionally 10KN, optionally 20KN, optionally 40KN, or have higher or lower or intermediate values, as for example DADCO Ultra Force™ Nitrogen Gas Spring type U.1600 with work forces of approximately 15KN-23.5KN. In an exemplary embodiment of the invention, gas spring 1141 is coupled to pressure pulse gun housing 1110, by fastening spring connecting nut 1142 to spring-gun thread 1111. Optionally, a plurality of different springs of different pressures and/or travel length are provided for selection by a user.

In an exemplary embodiment of the invention, distal movement of gas spring rod 1141b and/or pusher 1143 is prevented by stopper assembly 1130. Optionally, stopper assembly comprises a stopper 1131 which is slidably coupled to stopper guide 1133 and can selectively move inward or outward. When stopper 1131 is in inward position it can serve to prevent substantial distal movement of gas spring rod 1141b and/or pusher 1143. Optionally, said prevention is accomplished by engagement of stopper 1131 bottom end 1132 with e.g. pusher 1143 proximal end as shown in FIG. 1D. Optionally, rod 1141b and pusher 1143 are provided as a single part.

In an exemplary embodiment of the invention, trigger assembly 1120 incorporates trigger 1121, which is optionally rotatably coupled to housing 1110 by hinge 1122, stopper pin 1123 and sliding tooth 1124, lowing trigger 1121 to swivel. Stopper 1131 engages trigger assembly 1120 by stopper pin 1123, so that it can be slid by actuating trigger 1121. Optionally, when trigger 1121 distal end is pushed down, e.g. by finger pressing, it rotates clockwise around hinge 1122 (with respect to FIG. 1C outline), stopper pin 1123 forces stopper 1131 to slide upwardly while releasing its engagement with e.g. pusher 1143. In an exemplary embodiment of the invention, prevention of inadvertent triggering of trigger 1121 can be selectively maintained by using at least one safety apparatuses. Optionally, a clockwise rotation of trigger 1121 is prevented by a rotationally engaging trigger safety pin 1125, which can be manually rotated with trigger safety lever 1126. Optionally, said lever has two positions for locking and releasing trigger 1121 revolving, respectively. In an exemplary embodiment of the invention, when medicament cartridge 1200 is coupled to pressure pulse gun 1100, via gun-magazine thread 1112, an optional second safety mechanism is released, thus enabling trigger 1121 clockwise revolving. Optionally, coupling of medicament cartridge 1200 proximally slides cartridge-safety pin 1127, thus enabling inward movement of sliding tooth 1124 (e.g., clockwise rotation of trigger 1121).

FIGS. 1E-G describe an exemplary embodiment of medicament cartridge 1200. In an exemplary embodiment of the invention, cartridge 1200 generally consists of cartridge body 1210, plunger assembly 1220 and medicament reservoir 1230. In an exemplary embodiment of the invention, when activating pressure pulse gun 1100, which is coupled to medicament cartridge 1200, gas spring 1141 is activated so pusher 1143 is forced to engage and press plunger 1220 distally, thus forcing medicament previously stored in reservoir 1230 to expel towards hose 1300, injection head 1400 and to body (e.g., into treated live tissue).

In an exemplary embodiment of the invention, plunger assembly comprises plunger 1221, inner bore 1223 and unidirectional valve 1224. Optionally, cartridge 1200 is filled with medication prior to treatment and/or storage, by medical personnel or by the product provider. Optionally, valve 1224 can be operated (e.g. moved from opened to closed position or vice versa) by fluid pressure and/or by a mechanical pusher). In an exemplary embodiment of the invention, a syringe (e.g. a regular disposable medical syringe) equipped with a hollow needle (not shown) and pre-filled with a preferred medicament, is introduced into plunger assembly 1220 through inner bore 1223, until needle distal end is adjacent to unidirectional valve 1224 and/or reservoir 1230. Valve 1224 permits drug injection into reservoir 1230 and/or prevent any backflow. In an exemplary embodiment of the invention, the medicament is used to fill the entire volume of medicament reservoir 1230 and the inner volumes of hose 1300 and injection head 1400 (connected to medicament reservoir 1230 by reservoir outlet 1231), and may even slightly spill through it in order to expel trapped air. In an exemplary embodiment of the invention, the volume of medicament being urged into the drug delivery system is about 1 cc, optionally about 3 cc, optionally about 5 cc, optionally about 10 cc, optionally about 50 cc or higher or lower or intermediate value. In an exemplary embodiment of the invention, plunger shaft 1221, which is attached to plunger 1221, slides distally when pushed by pusher 1143, until plunger 1221 is pressed towards reservoir 1230 distal end and/or until a predetermined amount of medicament had been injected into body. In an exemplary embodiment of the invention, medicament reservoir 1230 volume, which may be substantially equivalent to the maximal medicament dose that can be injected to body, is about 0.05 cc, optionally about 0.1 cc, optionally about 0.2 cc, optionally about 0.5 cc, optionally about 1 cc, optionally about 2 cc, or higher or lower or intermediate value. In an alternative embodiment, the body of reservoir 1230 is of a self-sealing type and the injection using the syringe is through a wall of reservoir 1230.

In an exemplary embodiment of the invention, for this and/or other applications, the medicament injected includes Paclitaxel (commercially available as Taxol® by Bristol-Myers Squibb, N.Y., USA), Colchicine (available by Bedford Labs, Ohio, USA) and/or Triamcinolone (e.g. Kenalog® available by Bristol-Myers Squibb, N.J., USA). In an exemplary embodiment of the invention, a medication containing Taxol® in concentration of 0.1 to 1 mg/ml is applied for urological treatments. In an exemplary embodiment of the invention, a medication containing Taxol® in concentration of 0.5-0.8 mg/ml, optionally about 0.6 mg/ml is applied for treating the prostate region. In an exemplary embodiment of the invention, a medication containing Taxol® in concentration of 0.3-0.5 mg/ml, optionally about 0.36 mg/ml is applied for treating the urethra region (e.g., not prostate).

In an exemplary embodiment of the invention, hose 1300 is used to deliver pressurized medicament towards injection head 1400. Optionally, hose 1300 can resist inner pressures that exceed 1 atmosphere, optionally 10 atmospheres, optionally 100 atmospheres, optionally 500 atmospheres, optionally 1,000 atmospheres, optionally 2,000 atmospheres, or lower or higher or intermediate values. Optionally, hose 1300 is also flexible and/or bendable, thus permitting improved handling of the drug delivery system and its introduction into body. Optionally, the tube is between 5 and 50 cm long and between 1 and 10 or 20 mm in diameter.

In an exemplary embodiment of the invention, injection head 1400 (FIG. 1H) comprises a tip 1410, a body 1420, and a proximal end 1440, which is optionally designed to engage the distal end of hose 1300. Optionally, tip 1410 is sealed and designed to improve insertion of injection head 1400 into and through narrow hollow vessels. In an exemplary embodiment of the invention, body 1420 has plurality of micro-holes 1430 which serve as injection ports for the medicament. Optionally, micro-holes 1430 average diameter is in the range of 5-200 micrometers, optionally about 50 micrometers. The number, diameters, distribution and/or patterns of micro-holes can vary, for example, according to desired injection geometry, depth, and/or velocity.

An exemplary cocking instrument 1500 is described in FIG. 1I, and may comprise pusher 1510, which is threaded to an inner nut, rotating handle 1520 and grip area 1530. A cocking instrument may be needed to cock exemplary spring 1141, especially if pressure pulse gun 1100 is intended for multiple use. After cocking instrument 1500 is coupled to gun 1100 distal end (e.g. by fastening it to gun-magazine thread 1112), handle 1520 may be rotated (clockwise or counterclockwise) thus promoting proximal traveling of pusher 1510 towards pusher 1143 and gas spring 1141, until gas spring rod 1141b is pressed to a desired location and spring 1141 is cocked.

In an exemplary embodiment of the invention, pressure pulse gun 1100 and medicament cartridge 1200, separated or assembled together, can be handheld, e.g., have total weight and dimensions that enable relatively low strenuous and/or relatively comfortable handling by the operator. In an exemplary embodiment of the invention, the total weight of gun 1100 and cartridge 1200 is in the range of 0.2-2 Kg, optionally 0.3-1 Kg, optionally about 0.5 Kg, without medication.

FIG. 2 schematically describes an exemplary general method of treatment for restenotic urology vessels. Prior to drug injection (e.g., gun operation 2400), some preparations may be made, according to the device features and/or specific medical case. These preparations may include: area preparations 2100 and/or gun preparation 2200 and/or medicament preparations 2300. All three can be done in parallel and/or in series. The stenotic area may located 2120 with any known method—if by invasive or by non-invasive means, and this may also be dependant on the specific case (for example, if the stenosis is in the urethra or the prostate is to be treated through the urethra). It may be also necessary to drain 2110 the urinary bladder prior to treatment. When the area is located and ready for treatment, the physician can introduce injection head 1400 into the urethra and position it (e.g., the treatment holes 1430) 2130 adjacent to the stenotic area. In an exemplary embodiment of the invention, at least part of micro-holes 1430 should be directly beneath and/or in direct communication with the surrounding tissue needs to be treated, as the medicament will be injected as separate jets laterally projected from micro-holes 1430 and aimed to penetrate said adjacent tissue.

If pressure pulse gun 1100 is not cocked, cocking instrument 1500 (or any other cocking means) can be used to cock 2210 gas spring 1141 to a desired pressure/position, which will depend on the forces/pressures that should be applied. Optionally, when a gas spring is used, a foot pump or an electrical pump or a hand pump is used to pressure spring 1141.

Medicament preparation 2300 is optionally carried out, unless, for example, the drug delivery system had been provided fully assembled (or medicament cartridge attached to hose and tip) and already filled with medicament (e.g. in a case of optional single-use system). An optional step is to fill medicament cartridge 1200 with medicament, for example by using a syringe equipped with an appropriate adapter, as described above. If cartridge 1200 is not already attached to pulse gun 1100, then it is optionally coupled 2320 to thread 1112, as described above. Optionally, when attaching cartridge 1200 to gun 1100, cartridge-safety pin 1127 may slide proximally and/or otherwise allow trigger 1121 rotation (unless other safety means are active).

When all needed preparations are met the physician may advance to gun operation phase 2400. If any safety mechanisms (e.g., safety pin 1125) are still preventing trigger movement, the operator optionally releases 2410 them prior to medicament injection. Medicament injection is performed by operating 2420 trigger 1121, for example by pressing it (e.g., with the thumb). Trigger operation 2420 initiates a sequence of events by which the energy stored in the energy source (e.g. gas spring 1141) is transformed to a single impact that causes a high pressure pulse of the medicament. In an exemplary embodiment of the invention, at least two medicament streams are injected from injection head 1400 in order to penetrate the adjacent tissue. Optionally, a single medicament stream penetration depth is in the range of 0.05-15 mm, optionally 0.1-3 mm, optionally about 0.5 mm.

The above description focuses on a single shot medicament dose. For a second dose, an operator may replace or refill the cartridge and re-cock or replace the energy source.

The invention, in some embodiments thereof, further include other exemplary pressure pulse guns (not shown), that may be connected or comprise a plurality of cartridges and/or a magazine containing several dosages of medicament to be released separately by demand, and/or an automatic or semi-automatic refilling and/or re-cocking mechanism.

After medicament injection is fully performed, the physician may end (2430) the procedure by pulling injection head 1400 out of body.

In this and other embodiments, device parts may be attached, for example, by threading or by rotational snap-fitting, whereby a rotation is necessary to release the connection and the connection is maintained by an interference.

### Example 2

### Transmucosal Applications

In an exemplary embodiment of the invention, a pulse gun is employed to inject medications through mucous membranes to underlying cells. Some uses of drug delivery systems in Transmucosal treatments are described in International patent application number PCT/IL2006/000087 to Beyar et al, the disclosure of which is fully incorporated herein for reference.

In an exemplary embodiment of the invention, said pulse gun coupled to or as part of a drug delivery system, is used to perform anti-inflammatory therapy as in the case of treating Allergic Rhinitis. Optionally, said medications may comprise intranasal corticosteroids and/or antihistamines. Optionally, said medications may include: beclomethasone, budesonide, flunisolide propionate, fluticasone propionate, mometasone furoate, and triamcinolone acetonide. Optionally, the medications are delivered in aqueous preparations.

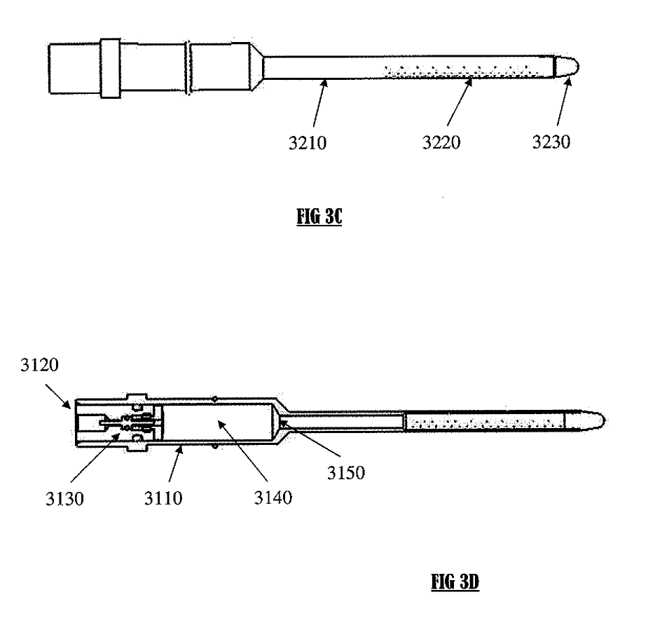

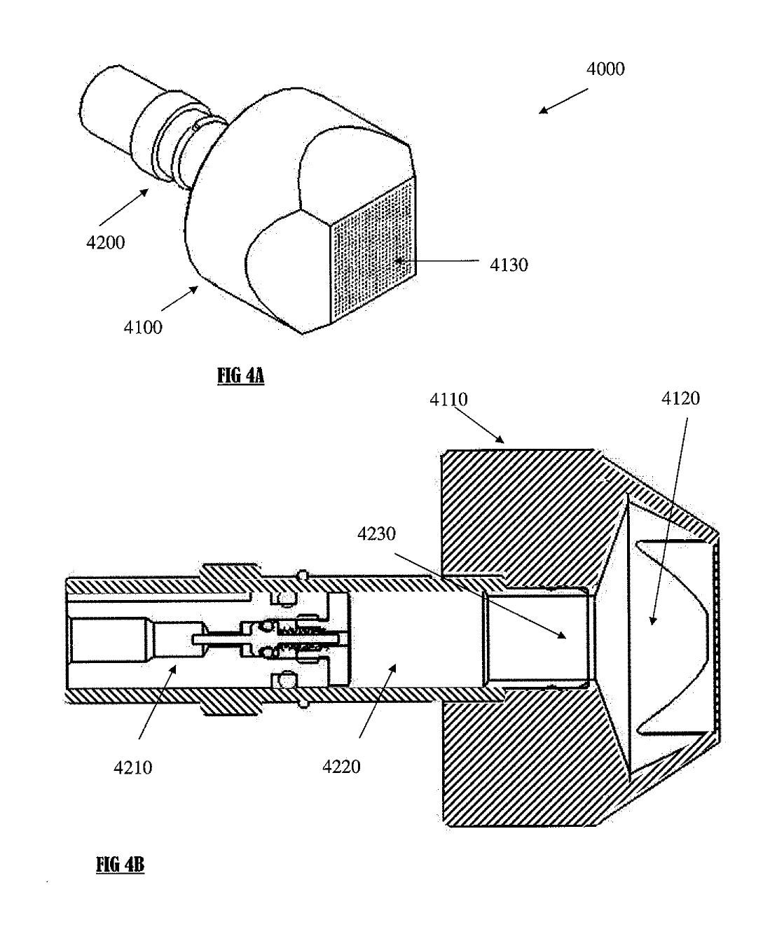

FIGS. 3A-3D describe an exemplary drug delivery system for transmucoasl applications. FIG. 3A illustrate a complete system that comprises pressure pulse gun 1100 (e.g., which may be the same as for the previous application) and Intra-Nasal Penetrating drug delivery system (or INAP system) 3000. An exemplary INAP system may comprise medicament cartridge 3100 and injection head 3200. Optionally, medicament cartridge 3100 and injection head 3200 are coupled or may be coupled directly one to the other or be produced or supplied as a single part (as illustrated in FIGS. 3B-3D). Alternatively, medicament cartridge 3100 and injection head are connected by a connecting element and/or adapter, as for example by a flexible tube or by a coupler, such as a threading.

Exemplary injection head 3200 may comprise body 3210, which is at least partially perforated by plurality of holes 3220, and of optional tip 3230. Body 3210 is optionally rigid enough in order to withstand manual manipulations within nostril and/or nasal sinuses for example when it is advanced to a desired location. In an exemplary embodiment of the invention, body 3210 is made of stainless steel and/or of any other biocompatible rigid material. In alternative exemplary embodiment of the invention, body 3210 is made of non-rigid and/or highly elastic material, for example rubber or plastic material. Optionally, several types of injection heads, which differ by body material and/or design, can be submitted to the physician who may pick the preferred one per specific application and/or patient.

In an exemplary embodiment of the invention, body 3210 distal end is at least partially perforated by plurality of holes 3220. Optionally, holes 3220 cover the entire circumference of at least a portion of a distal side of body 3210. In an exemplary embodiment of the invention, holes 3220 only partially covers such a portion, optionally about 180 degrees or 90 degrees of rotation (e.g., as illustrated in FIG. 3B), so medicament injection is more directional and submitted to a specific nasal region as preferred by the physician. Optionally, the INAP system contains a mark (e.g. engraving 3160) to indicate a perforation orientation. Optionally, holes 3220 have substantially similar design and/or dimensions. Alternatively, holes 3220 have changing design and/or dimensions along body 3210 longitudinal axis. In an exemplary embodiment of the invention, at least one hole of holes 3220 has a diameter of about 10 microns, optionally about 50 microns, optionally about 100 microns, optionally about 500 microns, optionally about 1 mm, or higher or lower or intermediate value. In an exemplary embodiment of the invention, holes 3220 pattern has a length that exceeds optionally 10 mm, optionally 30 mm, optionally 50 mm, optionally 100 mm, or lesser or higher or intermediate value. In an exemplary embodiment of the invention, holes 3220 pattern has a maximal width (e.g., partial or full circumference) that exceeds optionally 1 mm, optionally 3 mm, optionally 5 mm, optionally 10 mm, optionally 30 mm, or lesser or higher or intermediate value.

Exemplary medicament cartridge 3100 may comprise of body 3110, having an inlet 3120 and an outlet 3150, plunger assembly 3130 and medicament reservoir. In an exemplary embodiment of the invention, the design of cartridge 3100, including but not limited to plunger assembly 3130 and/or medicament reservoir 3140, is substantially similar to medicament cartridge 1200 of the previous exemplary embodiment.

Optionally, pressure pulse gun 1100 is set to produce enough pressure and/or force to eject medication via a plurality of jets, whereas at least one or some or all of said jets has a velocity capable of piercing mucous membranes to a preferred depth. Optionally, the target area is the sub-mucosa layer, also referred to as lamina propria, which is normally located from about 0.001 mm to about 2.5 mm deep. In an exemplary embodiment of the invention, at least one medication jet has a velocity capable of penetrating intra-nasal layers to depths of optionally about 0.1 mm optionally about 0.5 mm optionally about 1 mm optionally about 1.5 mm, optionally about 3 mm, or lower or higher or intermediate value.

### Example 3

### Exterior Tissue Treatment (Skin)

In an exemplary embodiment of the invention, a pulse gun according to the present invention is employed to inject medications or any other fluidic substances to treat exterior tissue, such as in cases of skin disorders, when a medicament or other fluids can be distributed for treating skin diseases or for cosmetic reasons. Dermatologic drug delivery systems may be used, for example, to inject cosmetic fillers (e.g. wrinkles fillers), as for example fat grafts, bovine collagen-based fillers, human collagen fillers, Hyaluronic acid fillers, hydroxylapatite fillers and/or Microdroplet Liquid Silicone. A second possible exemplary application may injecting minute doses of toxins for cosmetic use or other skin treatments, such as using Botulinum toxin (commercially known as Botox®, a product of Allergan, Inc., Irvine, Calif.) for softening frown lines between the eyebrows. A third option may be injecting absorbable and/or evaporating fluids, such as saline (also included in term “medicament”), to a wrinkled skin area for exterior tissue or in vivo skin rejuvenation and scar management treatments. Optionally, the saline triggers tissue regeneration. A forth option may be injecting anti fungal medicament to a relatively large skin and/or nail area. Other procedures which involve transdermal needleless (or needle-based) injections in the form of single or a plurality of fluid jets for the treatment of live tissues are also covered by this application and may be used with apparatus as herein (e.g., single or multiple stream needless injection. Optionally, the number of streams in this and/or other application is, 2, 10, 20, 40, 80, 200, 1000 or intermediate or a greater number of simultaneous streams, depending for example, on desired effect, medicament delivered and/or tissue type sand/or size being treated.

Some usage of drug delivery systems for dermatological treatment or other exterior tissue use is described in International patent application number PCT/IL2006/000087 to Beyar et al, the disclosure of which is fully incorporated herein by reference.

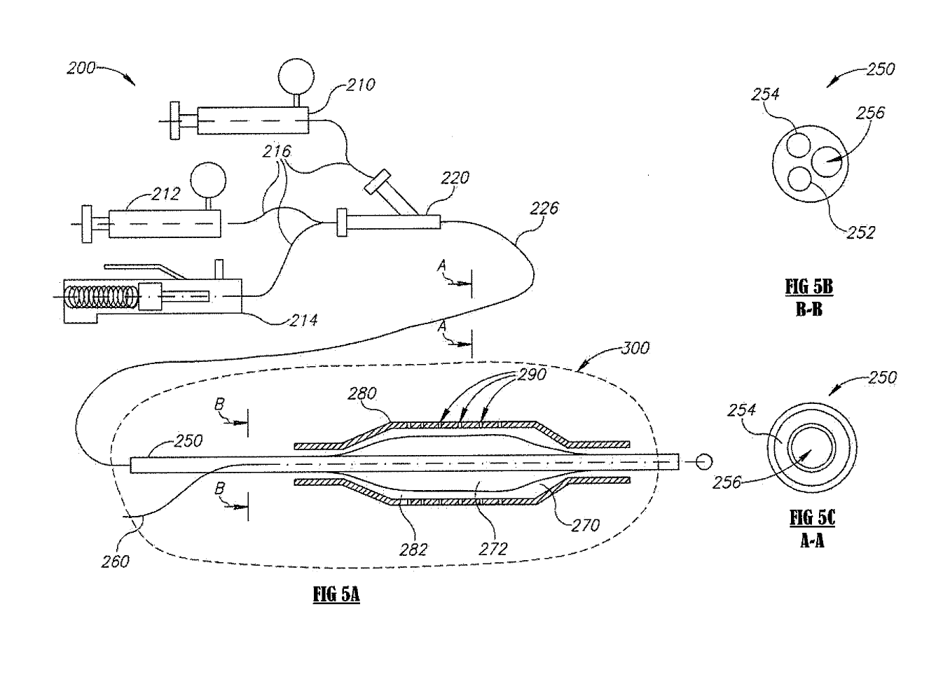

FIGS. 4A-4B respectively illustrate an isometric view and a cross section view of a fluid delivery system 4000 for exterior tissue use, comprising a pressure pulse gun (not shown, optionally as above), a fluid cartridge 4200 and an injection head 4100 having a plurality of injection ports 4130. In an exemplary embodiment of the invention, pressure pulse gun 1100, as illustrated in FIGS. 1B-1D, is used and can be coupled to cartridge 4200 in similar matter as to cartridge 1200. Optionally, cartridge 4200 comprises plunger assembly 4210 which is very similar to plunger assembly 1220 of cartridge 1200. Optionally, cartridge 4200 further comprising fluid reservoir 4220 with distal outlet 4230. Fluid may be introduced into reservoir 4220 in a similar manner to medicament filling 2310 of cartridge 1200, or it may be provided pre-filled.

Exemplary injection head 4100 is coupled (e.g. bolted) to fluid cartridge 4200 distal end. In an exemplary embodiment of the invention, different injection heads having different patterns, distribution and/or dimensions of ports 4130 are provided and may be selected by the operator according to different treatments and/or different areas of treatment; for example for injecting fluids to relatively large skin areas (as in the hips) or to relatively small delicate skin tissues (as facial skin). In an exemplary embodiment of the invention, a user can select a set that includes a pressure, a medicament and a nozzle pattern. Optionally, the set is provided as a plurality of each of pressure source, medicament container and nozzle. Optionally or alternatively, only one or two of the components are provided in a plurality of variants. Optionally, the pressure source is controllable to provide one of several pressure pulse forms and/or amplitudes.

FIG. 4A illustrates an exemplary rectangular pattern of ports 4130. Injection head 4100 further comprising body 4110 and injection nozzle 4120. When injection head 4100 is coupled over cartridge 4200, nozzle 4120 may serve as a channel for delivering the stored fluids towards ports 4130, when pressure pulse gun is activated. Similarly to medicament delivery system 1000, this system optionally produces a plurality of fluid streams through ports 4130, each having autonomous impact and velocity capable of penetrating into treated tissue layer(s).

### In-Vivo Blood Vessels Treatments

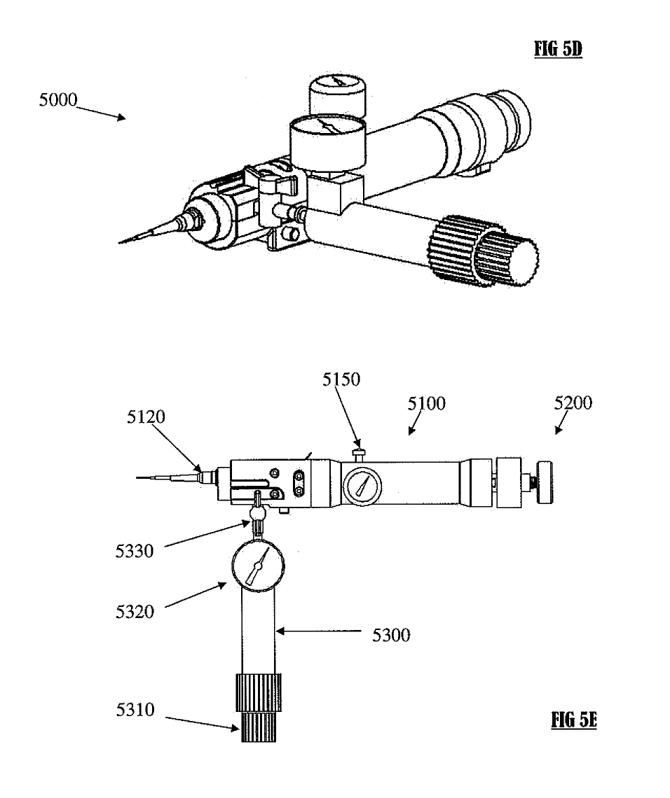

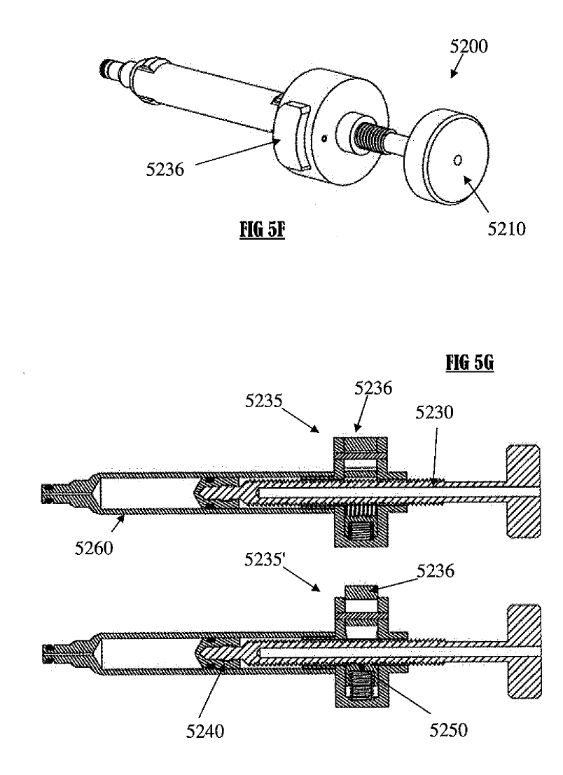

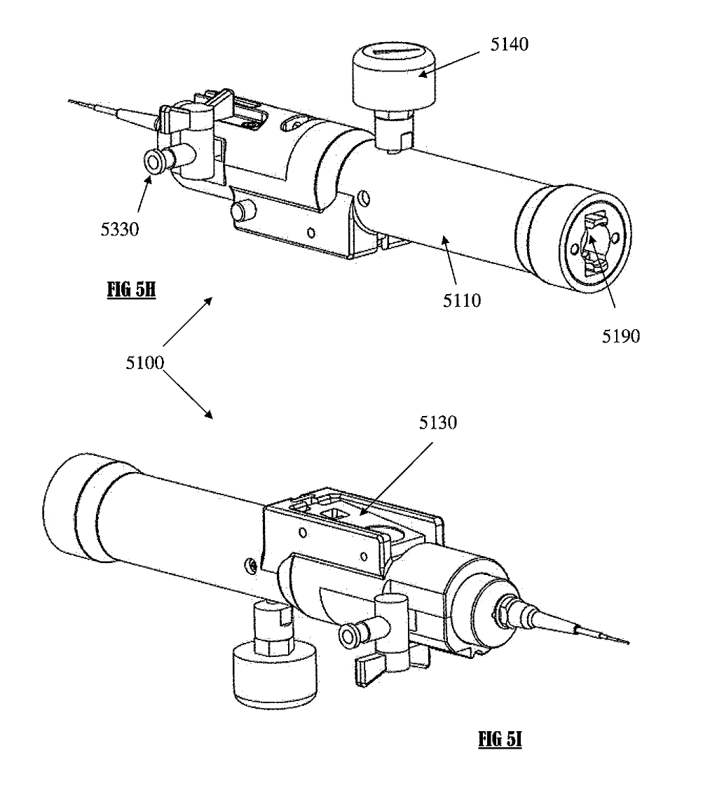

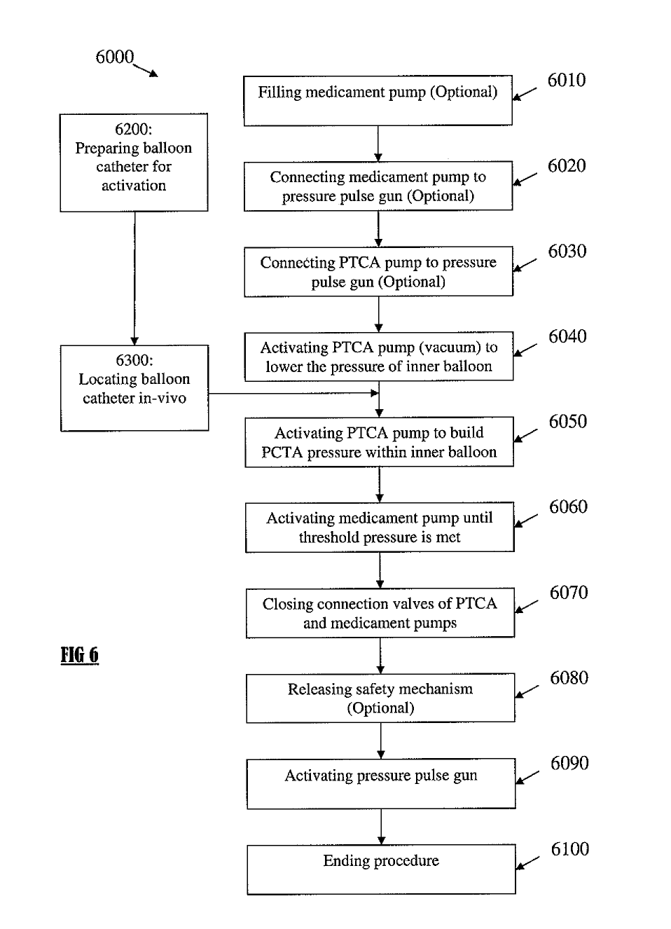

U.S. patent application Ser. No. 11/609,451 to Globerman et al, the disclosure of which is fully incorporated herein by reference, presents an exemplary drug delivery system for in-vivo treatment of bodily vessels (e.g. blood vessels) by injecting medicament into the vessels' wall tissues as a plurality of tissue penetrating jet streams. A general scheme for an exemplary system which may be used for carrying out such a process is illustrated in FIGS. 5A-C. System 200 is depicted as including a plurality of pressure sources, optionally three different pressure sources as pumps 210 and 212 and pressure pulse gun 214. System 200 further include a pressure sensitive valve 300 which is optionally a double chambered device characterized by an inner chamber and an outer chamber. Other components of system 200 may be provided to regulate internal pressures of the inner and outer chambers. In other exemplary embodiments of system 200, valve 300 is replaced by a valve with a different configuration.

In an exemplary embodiment of the invention, a medical procedure begins with insertion of valve 300 comprising outer balloon 280 and inner balloon 270 into a bodily vessel. Optionally, insertion is along a guide wire 260. In an exemplary embodiment of the invention, valve 300 is used to perform a PTCA as well as to inject fluid; optionally the insertion is to a site of stenosis.

After insertion to a desired site, inner balloon 270 is inflated. If PTCA is to be performed, inflation can be to a PTCA pressure. A PTCA pressure is typically in excess of 5, 10, 20 or 30 atmospheres. In an exemplary embodiment of the invention, pressure for inflation is provided by a pump 210 which pumps fluid via tubing 216 and/or connector 220 to lumen 254 of catheter 250 which is in fluid communication with lumen 272 of inner balloon 270. In an exemplary embodiment of the invention, pressure supplied by pump 210 is monitored, for example by a gauge on pump 210 and/or by a pressure sensor in balloon 270. Optionally, initial inflation can be to a PTCA pressure and pressure can be reduced for subsequent operation of valve 300 as an injector.

In an exemplary embodiment of the invention, inner balloon 270 expands and contacts an inner surface of outer balloon 280 sealing 130 holes 290. Optionally, balloon 270 is expanded to a degree which concurrently opens holes 290 (e.g. by stretching of outer balloon 280) and seals holes 290 (e.g. by covering). Valve 300 is now in a closed operational state.

In an exemplary embodiment of the invention, while valve 300 is closed, pump 212 delivers liquid medication via lumen 256 of catheter 250 to an entrance to inner lumen 282 of outer balloon 280 at a pressure slightly lower than maximal inflation and/or PTCA pressure. Optionally, lumens 256 and/or 282 are pre-filled (e.g. with medication) prior to insertion step. At this stage, inner balloon 270 continues to seal holes 290 of outer balloon 280 so that valve 300 remains closed.

In order to cause valve 300 to open, a pulse gun 214 applies a pressure pulse via lumen 256 so that pressure at the entrance to inner lumen 282 of outer balloon 280 increases to at least maximal inflation and/or PTCA pressure (e.g., the pressure inside balloon 270), and preferably to a substantially higher value. This immediate pressure increase causes fluid to flow into inner lumen 282 of outer balloon 280.

The pressure in lumen 282 of balloon 280 causes inner balloon 270 and outer balloon 280 to separate. Separation can result from contraction of inner balloon 270 (if it is sufficiently compliant) and/or expansion of outer balloon 280 (if it is sufficiently elastic). In an exemplary embodiment of the invention, contraction of inner balloon 270 uncovers at least some of holes 290 of outer balloon 280, so medication exits at high velocities. Because the medication is driven by a relatively high pressure, it can penetrate into a tissue of bodily vessel (e.g., coronary and/or peripherally or other blood vessels). The pressures described above are optionally chosen according to a desired penetration profile of the medication. It should be noted that, generally, the fluid leaving holes 270 leaves at about the pressure of the medicament, not at the pressure difference between the pressures in balloons 270 and 280.

FIGS. 5D-5I illustrate an exemplary drug delivery actuating system of the present invention which incorporates a pressure pulse gun and corresponds to the general scheme of drug delivery system 200 illustrated in FIGS. 5A-5C. Exemplary actuating system 5000 incorporates pressure pulse gun 5100 and pumps 5200 and 5300. Optionally, pumps 5200 and 5300 have functionality and capabilities which are substantially equivalent to regular PTCA pumps. Optionally, at lease one pump (e.g. pump 5200) is insertable and/or attachable to and/or provided as a single part with pressure pulse gun 5100. FIGS. 5I and 5H respectively illustrate pump 5200 and gun 5100 when they are not attached. Optionally, at least one pump (e.g. pump 5300) is releasably connectable to pressure pump 5100 or to any other element of actuating system 5000, for example by coupling it to luer lock 5330 prior to actuation.

Optionally, medicament pump 5200 is used for delivering medicament into valve 300, under a first predetermined pressure, which may serve as a threshold pressure prior to the high pressure pulse release, when gun 5100 is activated. Optionally, PTCA pump 5300 is used for inflating inner balloon 270 under a second predetermined pressure, which optionally may be substantially close to medicament threshold pressure described above. In an exemplary embodiment of the invention, said first predetermined pressure may be between 1 to 20 atmospheres, optionally 5-10 atmospheres, optionally about 8 atmospheres. In an exemplary embodiment of the invention, said second predetermined pressure may be equal or higher than first predetermined pressure, optionally is higher by 1 to 5 atmospheres, optionally by approximately 2 atmospheres. In an exemplary embodiment of the invention, the pressure pulse that may be released by gun 5100 exceeds 20 atmospheres, and is optionally between 50 to 700 atmospheres, optionally 80-200 atmospheres, optionally 100-150 atmospheres.

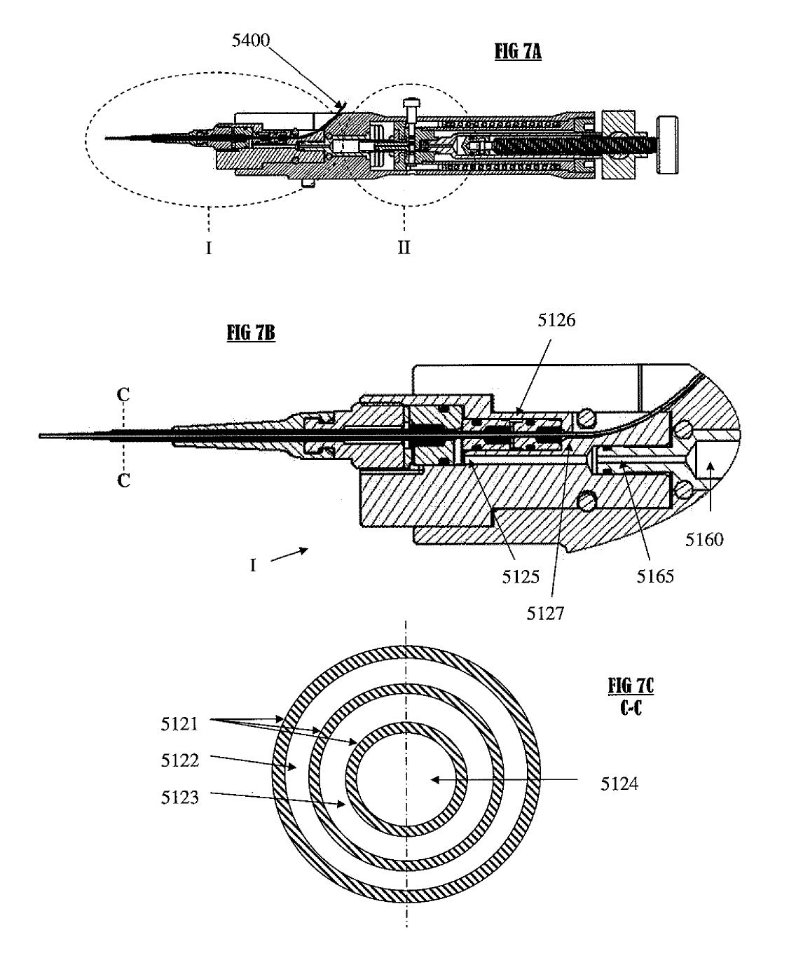

FIG. 6 shows exemplary sequential steps 6000 that may be undertaken to activate drug delivery system 200 in accordance with some embodiments of the present invention. Optionally, medicament pump 5200 is filled 6010 with medicament. In an exemplary embodiment of the invention, pump 5200 is a syringe-like device comprising medicament chamber 5260 and slidable piston 5240 (as illustrated in FIG. 5G). Optionally medicament is introduced into container 5260 by the operator from a separate medicament reservoir. Alternatively, pump 5200 is pre-filled with the medicament. In a similar manner, PTCA pump 5300 may optionally be filled with hydraulic fluid, as water or saline, but may preferably include contrast media.

In an exemplary embodiment of the invention, the medicament contains at least one of the following: Sirolimus/Rapamycin (commercially available as Rapamune® by Wyeth, N.J., USA), Zotarolimus (e.g. ABT-578 by Abbott Laboratories, Illinois, USA), Pimecrolimus (e.g., Elidel® by Novartis, Basel, Switzerland), Clobetasol (available by Dermovate, GlaxoSmithKline, Middlesex, UK) and Xyotax (available by Cell Therapeutic Inc, Seattle, USA). In an exemplary embodiment of the invention, the applied medication contains Rapamycin in concentration of 0.1-5 mg/ml, optionally about 1 mg/ml. Preferably, the Rapamycin content is in fluid state.

Optionally, for example in the case that pumps 5200 and 5300 are not delivered as integral parts of system 200, they are connected 6020 and 6030 (respectively) to it, optionally after they are filled and ready for use. Prior to insertion into body, pressure sensitive the balloon is optionally compressed (or provided compressed) to minimal dimensions to ease its travel within bodily vessels. Optionally, PTCA pump 5300 is first activated 6040 in a reverse order in order to lower the pressure of inner balloon 270. In parallel, other needed preparations of the balloon catheter (e.g., pressure sensitive valve 300 and/or catheter 250) may be further carried out 6200. For example, a medication lumen may be emptied of trapped air and an optional cover or seal should be taken out prior to use. After preparations, the balloon catheter may be introduced into body and be located 6300 adjacent to or in contact with a preferred area of a bodily organ.

After valve 300 is located 6300, PTCA pump 5300 is re-activated in order to build 6050 pressure within the inner balloon until a predetermined pressure is reached. In an exemplary embodiment of the invention, said inner pressure is between 2 to 50 atmospheres, optionally between 5 to 20 atmospheres, optionally is about 10 atmospheres. In an exemplary embodiment of the invention, said inner pressure is capable of performing PTCA.

In a next exemplary step, medicament pump 5200 is activated 6060 to introduce medicament into valve 300. Optionally, medicament is further pressurized by pump 5200 until a predetermined pressure is met. In an exemplary embodiment of the invention, said predetermined pressure is equal or lower to inner balloon 270 pressure as set, optionally is about 8 atmospheres.

Once valve 300 is fully charged, after steps 6050 and 6060 were performed, both pumps 5200 and 5300 (and/or pressure valves thereof) may be disconnected prior to pressure pulse gun 5100 actuation, for example, to avoid unnecessary energy loss. Optionally, connecting valves of both pumps (not shown) are closed 6070. Optional safety mechanism (not shown) may then be released 6080 and pressure pulse gun 5100 can be activated 6090 so a high pressure pulse is generated and forces pre-filled medication to expel out of valve 300 through perforated regions of outer balloon 280. Optionally, gun activation 6090 is accomplished by pressing triggering mechanism, such as trigger 5130 (illustrated in FIG. 5I).

In an exemplary embodiment of the invention, sequential steps 6050 to 6090 are performed relatively quickly in order to avoid complications. Optionally, said sequential steps should not exceed 30 seconds, optionally 10 seconds, optionally 5 seconds. Optionally, at least part of sequential steps 6050-6090 are performed at once, in parallel and/or in-series, for example by activating a single actuator. Optionally, steps 6070-6080 are performed automatically once step 6060 is selectively performed by the operator. Optionally, steps 6060-6080 are performed automatically once step 6050 is selectively performed by the operator.

The physician can then decide whether to end 6100 the procedure or to perform another injection in a same or in a different location. In order to remove valve 300 from patient or to move it to a different location, the operator optionally first deflates it to a desired minimal dimension. Optionally, this can be achieved by first substantially lowering the pressures of both medication and PTCA pumps, optionally to pressures lower than 1 atmospheres, and then to re-open pumps connection valves. Optionally, the relatively lower pressures “vacuum” excess fluids from valve 300 and deflate it accordingly.

The following sections will describe exemplary pump assemblies that incorporate a pressure pulse gun, which can be used in conjunction with the above drug delivery system, for treating peripheral and coronary blood vessels, respectively. It should be emphasized however, that same or similar devices can be used to treat other bodily vessels as well, optionally by implementing minor changes to them.

### Example 4

### Peripheral Blood Vessels Treatments

In an exemplary embodiment of the invention, a pulse gun according to some embodiments of the present invention is employed to inject medications in-vivo to treat blood vessels. FIGS. 5D-5I and 7A-8C an illustrate exemplary drug delivery actuating system 5000 specifically designed for treating bodily vessels areas located near the insertion point of the catheter into patient body. Optionally, system 5000 is designed for delivering medicaments to blood vessels walls through an over-the-wire catheter. In an exemplary embodiment of the invention, said bodily vessels are peripheral blood vessels, for example, in body appendages.

In an exemplary embodiment of the invention, pressure pulse gun 5100 incorporates body 5110, tip 5120, trigger 5130, medicament reservoir 5160, gun spring 5170 and pusher 5180. In an exemplary embodiment of the invention, medication pump 5200 includes knob 5210, bolted shaft 5230 distally connected to piston 5240, stationary nut 5250, chamber 5260 and outlet 5270. In an exemplary embodiment of the invention, PTCA pump 5300 is a commercially available PTCA pump that includes handle 5310. Optionally, pump 5300 further includes pressure meter 5320. Optionally, PTCA pump 5300 is substantially similar in design and/or functionality and/or includes several parts as in medication pump 5200. Optionally, pump 5300 is an integral part of pressure pulse gun 5100. Alternatively, pump 5300 is releasably coupled to gun 5100 through valve 5330. Optionally, valve 5330 is or includes a luer lock.

Optionally, gun body 5110 serves also as a housing for medicament pump 5200 and/or for PTCA pump 5300. Optionally, medicament pump 5200 is inserted into body 5110 distal end and is coupled to gun 5100 either by threading, bolting or simply by pressing it inside through opening 5190 of gun 5100 (that is illustrated in FIG. 5H). Optionally, pump 5200 has at least two modes of operation for building or lowering pressures: 5235 and 5235′, as illustrated in FIG. 5G. Optional first mode 5235′ allows piston 5240 to travel only by rotating knob 5210 and/or shaft 5230 with respect to stationary nut 5250. Optional second mode 5235 allows piston 5240 to travel by pulling or pushing knob 5210 and/or shaft 5230. In an exemplary embodiment of the invention, pump 5200 further includes button 5236 that is connected to nut 5250, and may be selectively switched from first rotational mode 5235′ to second pressing mode 5235 as described above. Optionally, button 5236 is a double mode push button having partially threaded bore, so in first mode 5235′ inner threading can engage bolted shaft 5230 and in second mode 5235 shaft 5230 can travel freely through the bore.

Optionally, pressure meter 5140 is coupled to gun 5100 or to pump 5200 and may be in direct communication (either selectively or not) with medication chamber 5260 of pump 5200.

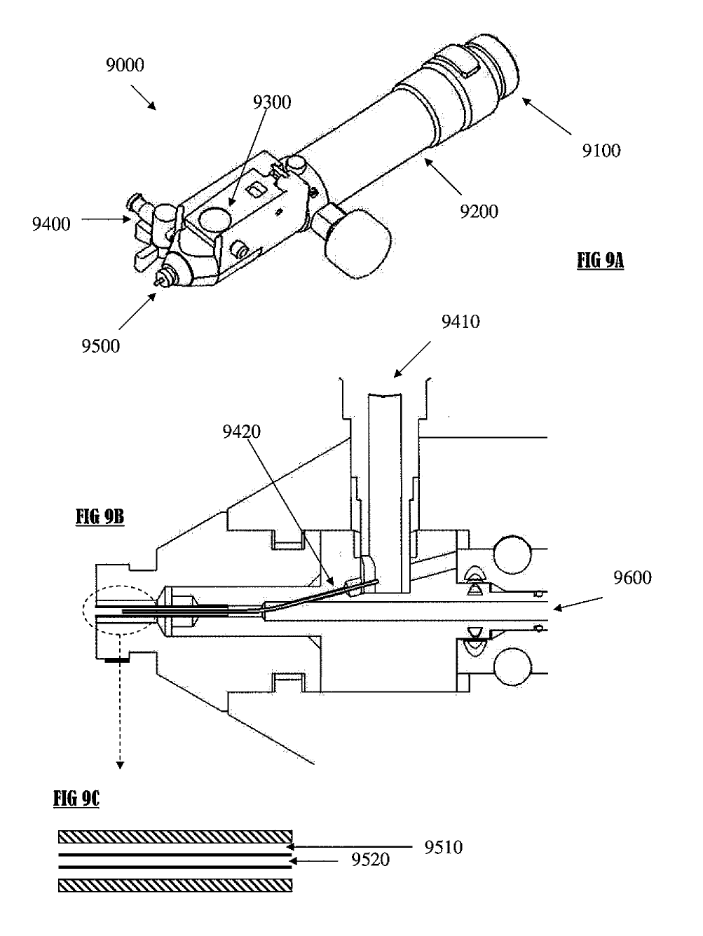

In an exemplary embodiment of the invention, medicament or any other fluid material may be selectively delivered from pre-filled medicament chamber 5260 towards gun 5100 medicament reservoir 5160 when piston 5240 is advanced distally. Optionally, medicament can travel through a relatively small diameter channel that includes pump outlet 5270 and jet 5165 that at least partially connect pump medicament chamber 5260 to gun reservoir 5160. Optionally, jet 5165 is a tube-like element having a relatively small inner diameter and relatively large outer diameter (as illustrated in FIG. 8B). Optionally, reservoir 5160 is contained within housing 5175, which is optionally, though not necessarily, tubular and having a substantially constant inner diameter.

In an exemplary embodiment of the invention, pusher 5180 is in contact with gun spring 5170. Optionally, pusher 5180 can travel distally when minimal preferred force is applied by spring 5170. Optionally, when spring 5170 is released from a predetermined compressed position, it applies force that moves pusher 5180 in a desired velocity, impact and/or distance, which are optionally defined by one or more of minimal, maximal and average values. Preferably, said velocity, impact and distance are pre-calculated to produce, at the exit from system 5000, a pressure pulse capable of injecting a preferred number of fluids jets capable of piercing into a bodily tissue up to a desired depth.

In an exemplary embodiment of the invention, pusher 5180 contains at least one groove 5135 that can be selectively engaged by trigger 5130. Optionally, a stopper element (not shown) is coupled to trigger 5130 and until the trigger is actuated (e.g. pressed), the stopper element engages groove 5135 so pusher 5180 is bounded to stationary mode. Optionally, when pusher 5180 is in stationary mode, spring 5170 is in a compressed, optionally fully compressed, position. Optionally, when trigger 5130 is actuated, spring 5170 is released.

Optionally, spring 5170 is a coil spring. Alternatively, spring 5170 is a gas spring.

In an exemplary embodiment of the invention, pusher 5180 includes plunger 5185 which is located on its distal side. Optionally, plunger 5185 is a hollow chamber with a substantially constant inner bore, having a proximal opening and a distal opening. Optionally, plunger 5185 is a hollow tube having an inner diameter and an outer diameter. In an exemplary embodiment of the invention, plunger 5185 is capable of containing jet 5165. Optionally, plunger 5185 inner diameter is equal or higher than jet 5165 outer diameter, and optionally they are substantially equal. Optionally, jet 5165 is stationary and serves as an inner core for plunger 5185 (i.e. plunger 5185 may selectively slider distally and/or proximally relatively to jet 5165). In an exemplary embodiment of the invention, plunger 5185 outer diameter is equal or lower than inner diameter of housing 5175 of medicament reservoir 5160 and they are optionally substantially equal. Optionally, housing 5175 is stationary and can serve as an outer core for plunger 5185. In an exemplary embodiment of the invention, plunger 5185 selectively travels from a first proximal position to a second distal position. Optionally, the first position is set by pusher 5180 position when spring 5170 is compressed and the second position is set by maximal distal travel of pusher 5180 after spring 5170 had been released. Optionally, pusher 5180 can travel distally until it is stopped by proximal edge of housing 5175.

In an exemplary embodiment of the invention, when plunger 5185 travels distally within medicament reservoir 5160, it pushes a volume of the medicament out of drug delivery actuating system 5000 and through tip 5120. In an exemplary embodiment of the invention, the plunger is a tube-like element having an inner diameter “ID” and an outer diameter “OD”, and is capable of sliding within a sleeve-like reservoir 5160 having an inner diameter substantially equal to plunger outer diameter, so when plunger 5185 travels within reservoir 5160 (or housing 5175) by distance “L”, it decreases reservoir 5160 inner volume by n*L/4*(OD²−ID²). Optionally, plunger 5185 travels into the sleeve only when the pressure pulse gun is activated, e.g., when spring 5170 is released. Optionally, reservoir 5160 is pre-filled with a medicament, so when the plunger travels into it, it expels a volume of medicament, which is substantially equal to π*L/4*(OD²−ID²). In an exemplary embodiment of the invention, the volume of medicament expelled is 0.05 cc, 0.1 cc, 0.2 cc, 0.4 cc, 1 cc, 3 cc, 5 cc, or higher or lower or intermediate amounts.

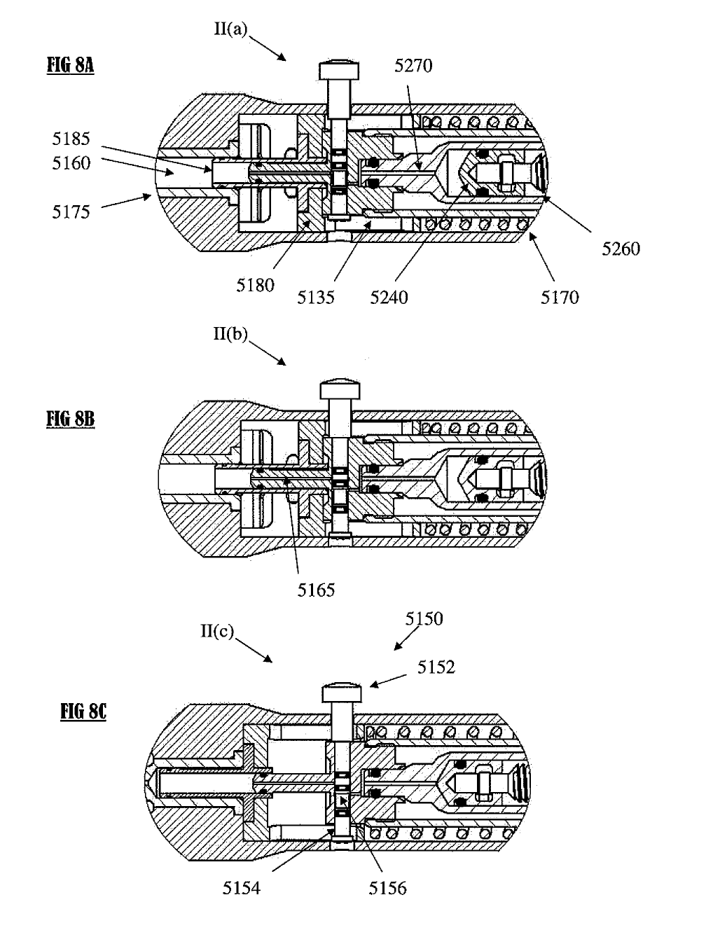

FIG. 7B is an exemplary zoom-in view of area “I” that is marked on FIG. 7A and includes distal end of pressure pulse gun 5100. FIG. 7C is an exemplary frontal cut-view of a catheter, made at imaginary line “C-C” illustrated in FIG. 7B; said catheter is coupled to or integral with tip 5120. In an exemplary embodiment of the invention, the catheter includes three lumens 5122, 5123 and 5124 that are divided by catheter walls 5121. Optionally, the three lumens are concentric, as illustrated in FIG. 7C. In an exemplary embodiment of the invention, a first lumen serves as a channel for guidewire travel, a second lumen serves for channeling hydraulic fluid for inner balloon 270 inflation and/or deflation, and a third lumen serves for channeling medicament towards pressure sensitive valve 300. Optionally, innermost lumen 5124 is the guidewire lumen, median lumen 5123 is the inflation/PTCA lumen and outermost lumen 5122 is the medicament delivery lumen. In an exemplary embodiment of the invention, guidewire 5400 is inserted into lumen 5124 through opening 5127 that is located on tip 5120 distal part. Optionally, hydraulic fluid (e.g. saline and/or contrast media) in delivered into lumen 5123 by opening 5126 that is located on tip 5120 and is in direct communication with valve 5330 that is connectable to PTCA pump 5300. Optionally, when valve 5330 is opened and connected to PTCA pump 5300, and when PTCA pump is actuated to build a predetermined pressure, the pressurized hydraulic fluid travels towards inner balloon 270 through lumen 5123. Optionally, if valve 5330 is closed while positive pressure is set, said pressure will be substantially maintained within lumen 5122 and/or inner balloon 270.

In an exemplary embodiment of the invention, pressure pulse gun 5100 further include a valve 5150 having at least two operational modes: a first mode in which medicament or any other fluid material can travel from medicament pump 5200 through the valve to jet 5165 and a second mode in which the valve is substantially or completely sealed for such travel. In an exemplary embodiment of the invention, valve 5150 includes body 5154, button 5152 and a non-sealed volume 5156. Optionally, the non-sealed volume is bounded by sealing element(s), e.g. at least two spaced O-rings. Optionally, valve 5150 can be situated in at least two general positions: a first “opened position” in which non-sealed volume 5156 provides direct fluid communication between medicament pump outlet 5270 and jet 5165 and a second “closed” position in which a sealed segment of valve 5150 prevents such fluid communication.

Area “II” marked on FIG. 7A is focused on an exemplary actuating mechanism of pressure pulse gun 5100. Said exemplary actuating mechanism includes, for example, at least three main positions II(a), II(b) and II(c) as illustrated on FIGS. 8A-8C (respectively). In position II(a) valve 5150 is in “opened” position, spring 5170 is optionally compressed and pusher 5180 is stationed in first proximal position and is optionally limited to that location by trigger 5130 and/or stopper element (not shown). In this position, exemplary steps 6040 to 6060 can be performed so medicament can be freely delivered into reservoir 5160 and/or catheter 250 under predetermined pressures. In position II(b) valve 5150 is in “closed” position, while both spring 5170 and pusher 5180 are in same position as in II(a). This position corresponds to steps 6070 and 6080 in which valves are closed and an optional safety mechanism is released. In position II(c) valve 5150 is in “closed” position, spring 5170 is substantially fully released and pusher 5180 is in second, preferably outermost, distal position. This position correlated to gun 5100 situation after step 6090 had been performed, e.g., gun 5100 was activated to produce a high pressure pulse that preferably outcomes with plurality of medication tissue piercing jets as described above.

### Example 5

### Coronary Blood Vessels Treatments

In an exemplary embodiment of the invention, a pulse gun according to the present invention is employed to inject medications in-vivo in order to treat coronary blood vessels. FIGS. 9A-9B illustrate an exemplary drug delivery actuating system 9000 specifically designed for treating bodily vessels in areas located relatively distant to the insertion point of the catheter into patient body. In an exemplary embodiment of the invention, said bodily vessels are coronary blood vessels. Exemplary system 9000 includes pressure pulse gun 9200, which combines tip 9500, trigger mechanism 9300 and optional valve connection 9400 (e.g. luer lock) intended for optional connection to an auxiliary element such as a PTCA pump. Optionally, system 9000 further includes medicament pump 9100, which is illustrated in FIG. 9A in nested position within gun 9200.

In an exemplary embodiment of the invention, actuating system 9000 is substantially or exactly the same in design and functionality with respect to actuating system 5000, so the description above can be at least partially implemented with system 9000. Optionally, exemplary sequential steps 6000 can be at least partially performed by system 9000. In an exemplary embodiment of the invention, systems 9000 and 5000 differ in tip design. Optionally, system 9000 tip does not include guidewire opening and/or accessibility as in the case of system 5000, for example when it is used in a rapid exchange style, as usually in the case of coronary blood vessels treatment.

FIG. 9B illustrates a lateral cut-view of system 9000 distal end. FIG. 9C is a schematic magnification for the cut-view of system tip 9500 distal end. In an exemplary embodiment of the invention, tip 9500 is designed to be coupled to a multi-lumen catheter, as catheter 250. Optionally, tip 9500 includes at least two lumens 9510 and 9520, optionally concentric (i.e. lumen 9520 is the inner lumen). Optionally, lumen 9510 is intended for medication delivery into a first lumen of said catheter and lumen 9520 is intended for hydraulic fluid (e.g. saline and/or contrast media) delivery into a second lumen of said catheter (e.g. for PTCA inflation).

In an exemplary embodiment of the invention, medication or other fluid material is provided to lumen 9510 through channel 9600, optionally due to a pressure applied by medicament pump 9100 and/or triggered gun 9200. In an exemplary embodiment of the invention, hydraulic fluid or other fluid material is provided to lumen 9520 through straw 9420 that is in direct fluid communication with port 9410 of valve 9400, optionally due to pressure applied by a PTCA pump coupled to valve 9400.

### Exemplary Medicament Preparation

In an exemplary embodiment of the invention, the medicament is Rapamycin and is prepared as follows:

(a) gas sterilize, (e.g., using ethylene oxide) 70 mg of Rapamycin powder, Wyeth Pharmaceuticals, Collegeville, Pa., U.S.A

(b) prepare 10 cc of a mixture of 15% TWEEN-80 in 100% ethanol. Sterilize, e.g., using gamma radiation.

(c) In a sterile work zone (e.g., clean room, clean chamber, operating room), mix 0.1 cc of mixture with all the powder. Mix in a vortex mixture for about 2 minutes until a transparent solution is achieved.

(d) Add 4.9 cc saline and mix gently, optionally to avoid inclusion of air bubbles. Final concentration 1.4 mg/ml.

In an exemplary embodiment of the invention, the medicament is Taxol and is prepared as follows:

(a) a 6 mg/ml concentration of a Taxol solution is obtained, for example, Medixel by Medison Pharma, Ltd, Israel;

(b) in a sterile zone, 4 mg of gas sterilized Taxol powder are added per ml of Taxol solution.

(c) the resulting 10 mg/ml Taxol solution is diluted using saline to a concentration of 1.25 mg/ml (e.g., a 1:7 ratio). Optionally, the above also reduces concentration of alcohol and chloroform (or other additives/preservatives) using in the Taxol solution down to ˜6.25% or less, more suitable for human in-vivo use.

Optionally or alternatively, ultra-flirtation sterilization and/or gamma sterilization are used instead or in addition to gas sterilization.

Optionally, a kit is provided which includes concentrated solutions (pre-saline), as saline may reduce shelf life.

### Exemplary Coronary System

In an exemplary embodiment of the invention, a coronary system includes a medicament injector, a pulse source, a PTCA pump, a catheter adapter and a catheter with a balloon-valve at its tip.

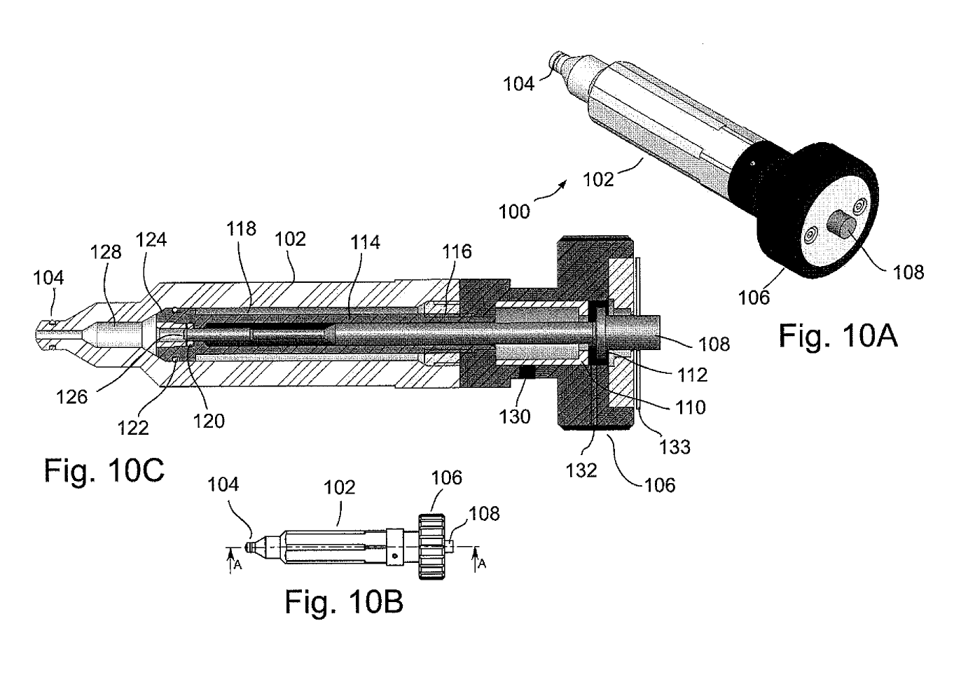

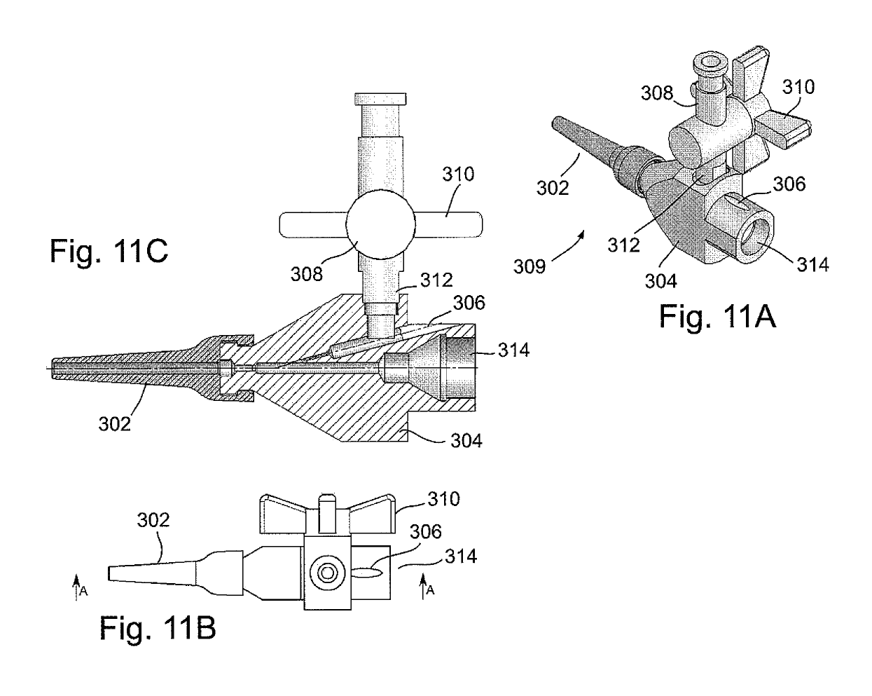

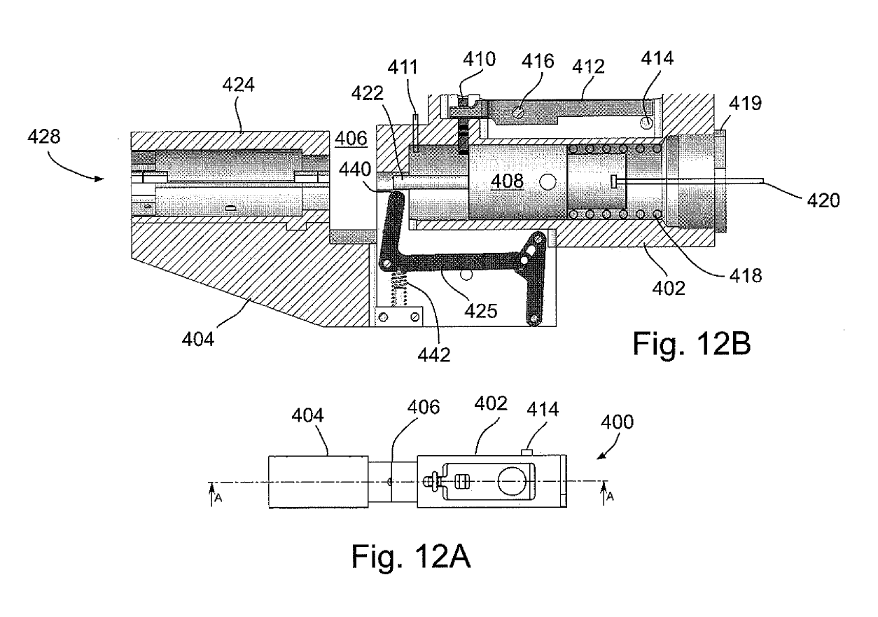

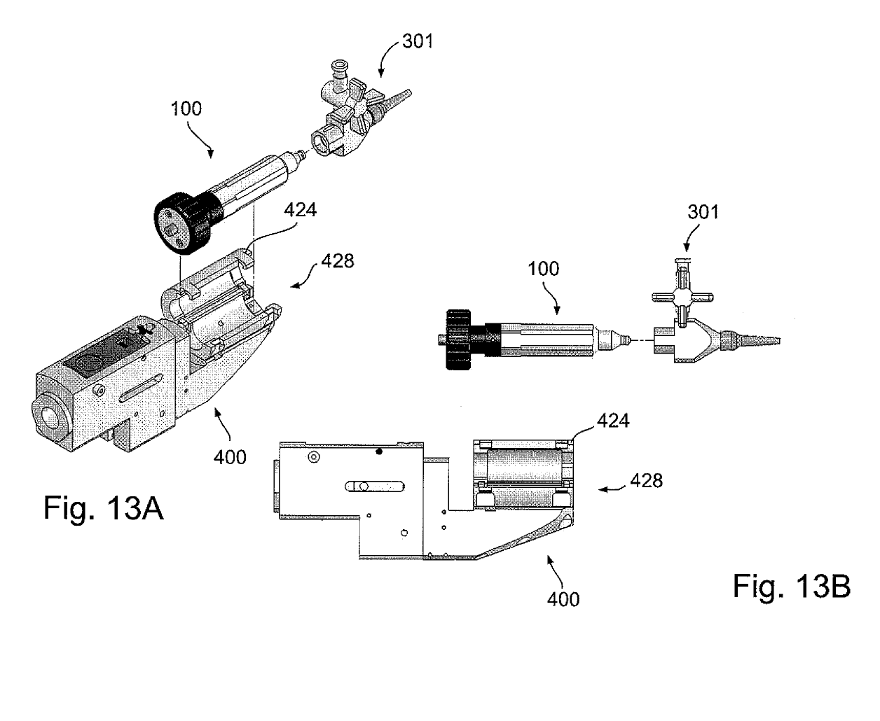

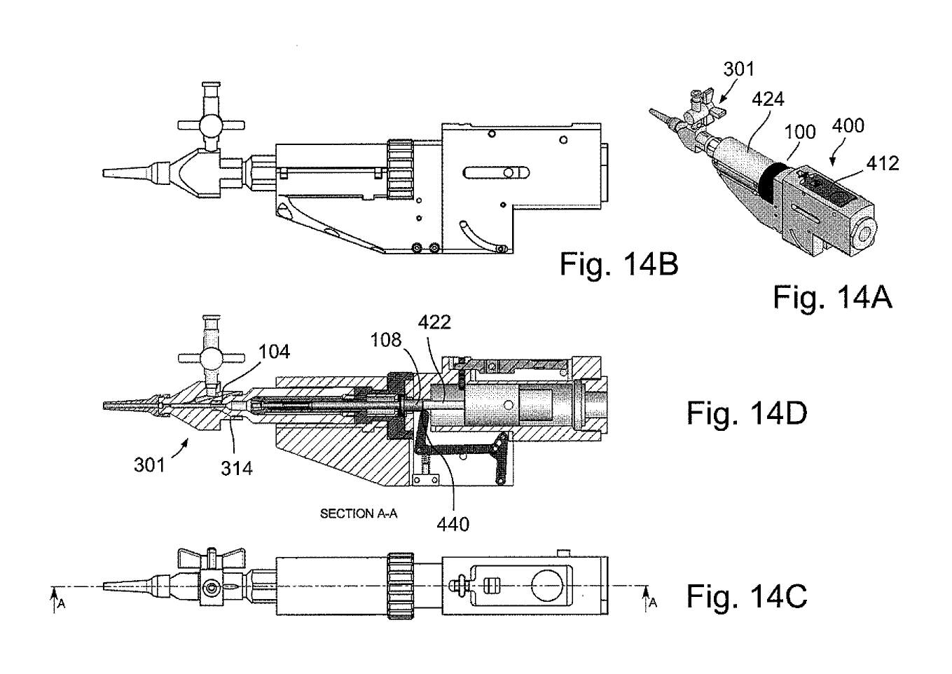

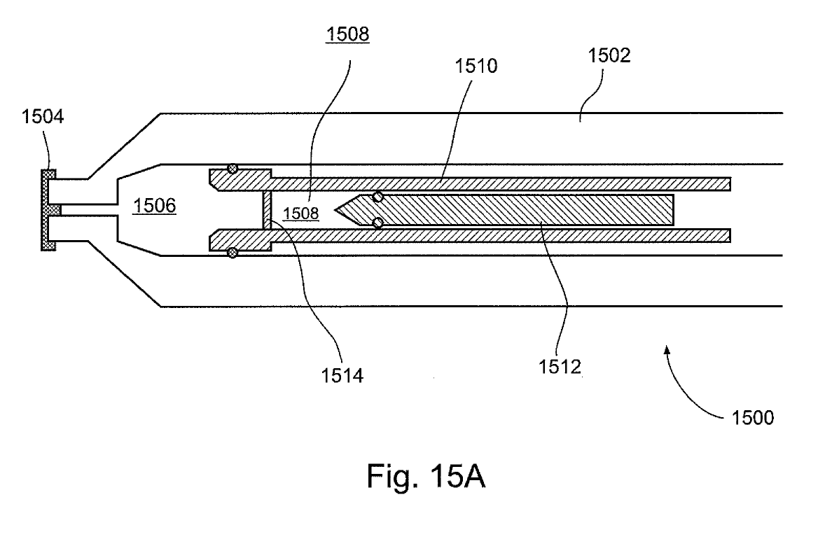

As will be described in greater detail below, FIGS. 10A-10C show a medicament injector 100; FIGS. 11A-11C show a catheter adapter 301 which is optionally integrally formed with or coupleable to a catheter (not shown); and FIGS. 12A-12B show a pulse source 400. FIGS. 13-14 show exemplary assembly of the system, not including a catheter and a PTCA pump.

In use, the following process is optionally used:

(a) Filling injector 100, optionally after dilution of the medicament. Optionally, injector 100 and the catheter are provided assembled and pre-filled. Alternatively, the medication is sucked into injector 100, by retracting a piston thereof. Thereafter, the piston may be advanced to remove air from the injector.

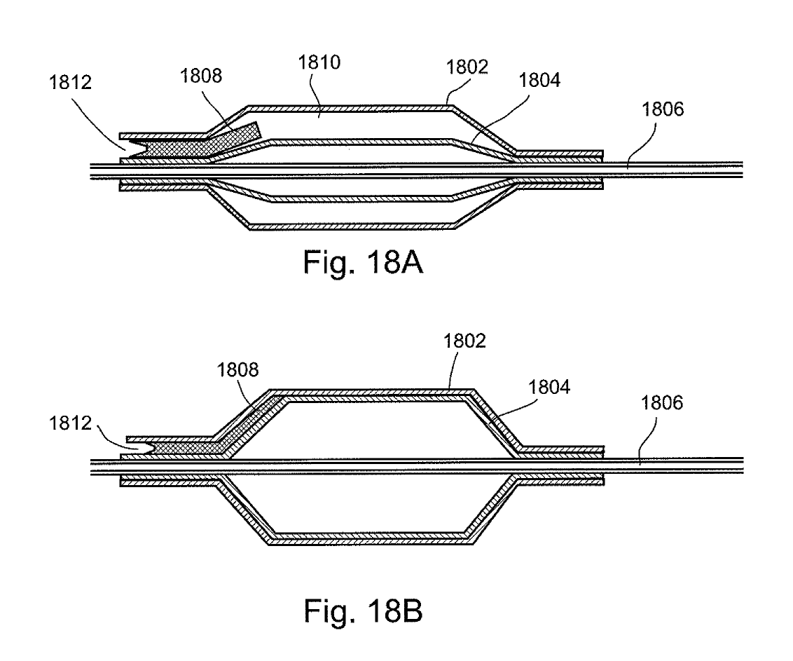

(b) The injector is attached to adapter 301 and a catheter and medicament is advanced into the catheter. Optionally, a distal air port is provided in the catheter (see FIG. 18) and/or medicament delivery pores in the balloon are used for air release. In an exemplary embodiment of the invention, the distal air port comprises a flexible tube (e.g., ID 0.2 mm polyethylene) which is later compressed by inflation of the inner balloon and thus sealed. Optionally, this prevents clogging of the pores by the medicament and/or allows a greater cross-section for air exhaust.

Referring specifically to FIGS. 18A and 18B, there is shown a balloon valve 1800 including an inner balloon 1804 and an outer balloon 1802 which includes a plurality of pores (not shown). A space 1810 is defined between the two balloons. A drainage tube 1808 is shown draining space 1810, to an outlet 1812 which is outside the valve. An optional guide wire channel/inflation channel 1806 is shown as well. When fluid pressure is applied only to balloon 1802, excess air and/or fluid can leak out through tube 1808. However, as shown in FIG. 18B, once inner balloon 1804 is inflated, tube 1808 is compressed and cannot leak medicament. Optionally, when additional pressure is applied to space 1810, the space does not open enough to allow tube 1808 to unblock. Optionally or alternatively, tube 1808 is compressed between forward sections of the balloons 1802 and 1804, where the increased pressure does not reach.

(c) Advance the catheter to the treatment area. Optionally, the catheter is not yet connected to the pulse source, which can reduce weight and increase ease of handling.

(d) Mount injector inside pulse source.

(e) Attach PTCA pump to catheter adepter and inflate inner balloon (e.g., about 12 bar in coronary vessels and about 9 bars in peripheral vessels). Optionally, this is performed before (d).

(f) Activate injector so that a pressure of medicament is raised to near (e.g., within 1, 2, 3 or 4 bar) that of the inner balloon. Optionally, the inner balloon pressure is reduced first. (g) Generate a pulse. Optionally, the pulse source is cocked before use, for example, by retraction of a piston thereof

(h) remove catheter (e.g., reduce pressures in medicament and in PTCA pump) and/or apply additional pulses (optionally refilling injector).

Referring specifically to FIGS. 10A-10C, where an exemplary injector 100 is shown. Injector 100 includes a body 102 having a hollow 128 for storing medicament (and optionally hollow 118 which may be in fluid communication with it, at least for part of travel of the pistons), a knob 106 or other means for manually or otherwise increasing medicament pressure (e.g., using an electric motor), a piston 108 for applying a pulse of pressure to the medicament and a top 104 for engaging a catheter adapter and allowing medicament flow therethrough.

FIG. 10C shows a cross-sectional view of injector 100, where a pre-pulse pressuring piston 114 is fully advanced and sealed against an inside surface 124 of body 102. A sealing ring 122 optionally seals the piston to body 102.

Tip 102 shows an optional sealing ring 104.

An optional sealing ring 120 is shown on a forward tip of piston 108.

During pre-pressuring, knob 106 is rotated, for example, manually, or by a motor (not shown), so that a threading on piston 114 engages a threading 116. Other means of converting rotational to linear motion, with gain, may be used. Piston 114 is rotatable. Alternatively, a pin, not shown, maintains its orientation. In general, such advancing of piston 114 will case retraction of piston 108. While shown as being concentric, pistons 108 and 114 may be side by side pistons. Optionally, piston 114 is larger in diameter, to enable a shorter syringe to provide a needed volume of medicament. Piston 108 optionally has a relatively small tip 126, to better apply a pulse to the medicament.

In pulse mode, piston 108 is advanced. Optionally, the amount of advance is limited by a limiter 110 which interferes with a widened section 112 of piston 108. Alternative designs, such as a limiter which lies within piston 108, may be provided (not shown).

Optionally, the amount of movement and thus pulse parameters and/or medicament volume moved, are controlled by modifying movement of piston 108. In one example, a control 130, for example, a thumb wheel or a set of pins matching apertures in limiter 110, allow user setting of the position of limiter 110. Optionally or alternatively, one or more spacers 132 may be placed through knob 106 into a space between limiter 110 and 108. Optionally or alternatively, one or more spacers 133 may be placed outside of knob 106, where they selectively engage an axial slot (not shown) in piston 108, and limit its extent of motion to the length of the slot.

A potential advantage of using a piston within a piston is that the use of a larger (outer) piston allows the injector to be relatively shorter for the desired dosage (e.g., 2 cc).

A potential advantage of using a piston within a piston is that the use of a smaller (inner) piston allows a better hydraulic amplification to be provided by the pulse gun.

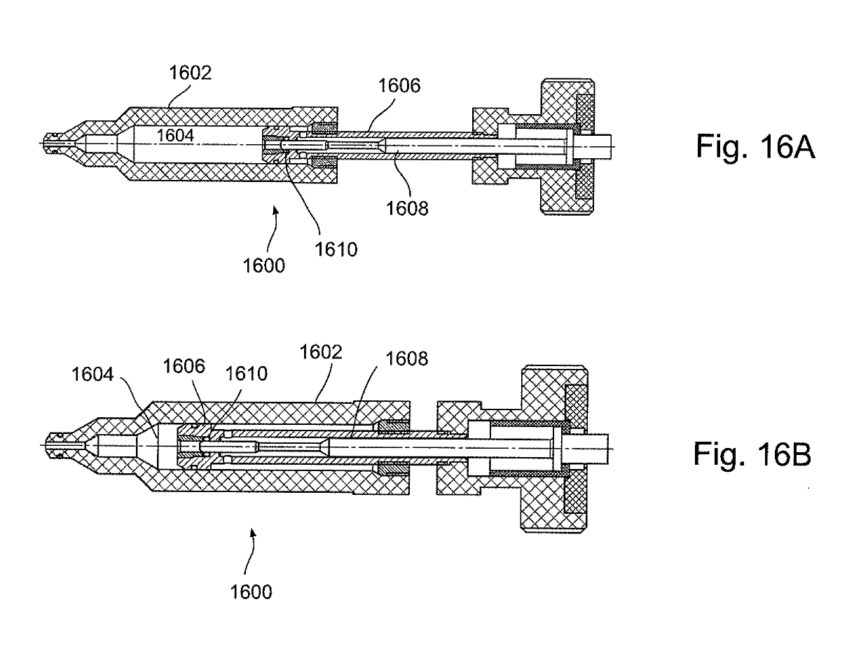

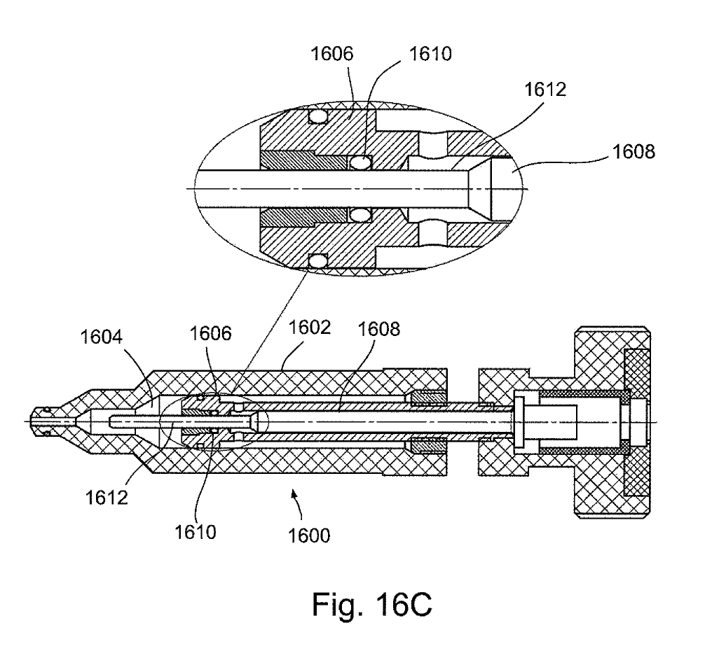

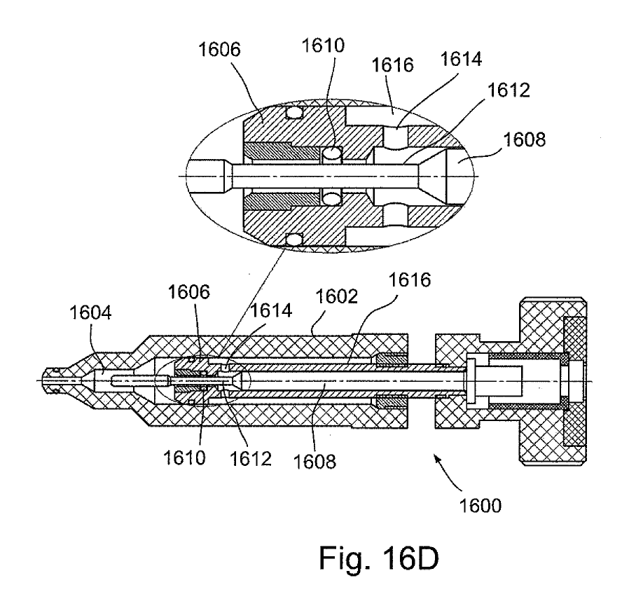

Referring specifically to FIGS. 16A-16D, there is shown a method and apparatus of immediately reducing the pressure when a pressure pulse is completed, possibly reducing leakage of medicament into surrounding tissue and/or blood, other than a desired target area.

FIG. 16A shows an injector 1600, including a body 1602, a medicament volume 1604, a first piston 1606 and an inner, pressure pulse piston 1608. A sliding seal 1610 seals the two pistons. FIG. 16A shows the injector before pressurizing and/or before medicament filling.

FIG. 16B shows injector 1600 after pressurizing and before use. The pistons are sealed to each other.

FIG. 16C shows injector 1600 after piston 1608 is advanced, with the modification (over FIGS. 16A, 16B and 16D) that piston 1608 has a uniform diameter in a section 1612 thereof adjacent seal 1610. As shown, there is no leakage of pressure form volume 1604, except out of the tip of the injector.

FIG. 16D shows injector 1600 after piston 1608 has been used, in a variant, where a section 1612 has a reduced diameter. As shown, this allows leakage of pressure past seal 1610, and optionally through apertures 1614 defined in piston 1606 and optionally into a hollow 1616 defined between piston 1606 and body 1602. Leakage past the seal is allowed as soon as piston 1608 is sufficiently advanced (which happens during the pulse application), thereby suddenly cutting off the pressure in medicament volume 1604. This feature may be provided in non-coronary applications as well. It should be noted that further shaping of the pressure pulse is optionally provide dif a balloon valve such as shown in FIG. 5 is used.

Referring specifically to FIGS. 11A-11C, a catheter adapter 301 is shown. An actual catheter may be attached to or be integral with a tip 302 thereof. An optional guide wire opening 306 is shown, for insertion of an optional guide wire. Optionally or alternatively, this opening is sealed and a guidewire, if any is provided side by side with the catheter (e.g., over the wire or rapid exchange). Optionally, a separate guide wire lumen (not shown) is provided in adapter 301 if the PTCA balloon used does not support sharing of a same lumen by the guidewire and inflation fluid. Pressure for the inner PTCA balloon is optionally provided via a valve 308, which is optionally closable using a stop-cock 310. Valve 308 is optionally screwed into a body 304 of adapter 302, using a threading 312. Alternatively, other attachment methods may be used, such as welding and rotational snap fitting.

A socket 314 is optionally adapted to receive and lock to top 104 of injector 100.

Optionally or alternatively, to a valve, a narrowing in the fluid pathway to the inner balloon (e.g., inside vale 308) may be provided, to reduce pressure pulse reforming by pressure escaping through valve 308.

In an exemplary embodiment of the invention, stop-cock 310 and/or other stopcocks are used to prevent pressure leakage, for example, by selectively disconnecting pressure gauges and/or pressure sources prior to pulse application. Optionally or alternatively, one-way valves are used, so loss is reduced. Optionally or alternatively, a fluid passage narrowing is used to reduce pressure loss.

Referring specifically to FIGS. 12A and 12B, a pulse gun 400 is shown. In the design shown, a pulse applying trigger 412 is an upper trigger activated by pressing. However, other trigger types and/or positions may be used. In the embodiment shown, a piston 408 is advanced by a spring 418 (while a mechanical spring is shown, a gas spring may be used), when an interfering pin 410 is moved away by rotation of trigger 412 around a pivot 416. Spring 412 is optionally cocked using a pull-rod 420 which optionally threads into piston 408. Optionally, a motor (not shown) coupled to rod 420 is used. Optionally or alternatively, rod 420 rotates around its axis to pull back piston 408. Optionally, a lever (not shown) is used to provide mechanical advantage to rod 420. Optionally, spring 418 is maintained in place in side a body 402 of gun 400, using a spring retainer 419, optionally attached by threading. Optionally, rotating retainer 419 modifies a compression of the spring and thus a pulse parameter.

In an exemplary embodiment of the invention, one or more safety mechanism are provided. One such mechanism is a pin 414 which prevents movement of trigger 412 until desired. Another exemplary safety mechanism relates to allowability of movement of piston 408. Generally, it is not desirable for piston 408 to advance of the injector is not loaded and/or if the injector is not pre-pressurized. As noted above, pressurization of injector 100 typically causes extension of piston 108. In an exemplary embodiment of the invention, a lever is provided which senses the position of piston 108. In the example shown, a lever 425 is provided which is elastically biased (e.g., using a spring 442) in a manner which causes forward movement of the lever towards piston 108 and which is pushed back by piston 108. Optionally, spring 442 is selected and/or settable to reflect various injector pressures.

In an exemplary embodiment of the invention, lever 425 is coupled to a safety pin (such as pin 414) preventing movement of trigger 412 unless injector 100 is properly pressurized.

In an electronic embodiment, a pressure sensor on lever 425 or on injector 100 can generate an indication to move pin 414 and/or to allow responding to pressing of lever 412. Optionally, an electronic control embodiments includes one or more of circuit(s) (e.g., a microcontroller), a battery (or other power source), one or more sensors (e.g., pressure sensors), one or more indicators (e.g., LED, graphic display and/or sound device) and/or one or more actuators (e.g., motors, linear actuators).

In an exemplary embodiment of the invention, movement of piston 408 can be limited using rod 420 (e.g., using a motion limiter on the rod) and/or using one or more spacers 411 placed in a path of movement of piston 408.

An extension 422 of piston 408 optionally contacts piston 108 and applies the pulse, in use. Optionally, a space 406 is provided to receive knob 106. Other geometrical designs may be provided, depending on the shape of injector 100, for example.

An injector holder 404, with a closable door 424 is optionally provided for holding body 102 of injector 100. When door 424 is closed, a cylindrical lumen 428 for receiving body 102 is created.

FIGS. 13A-14C show the assembly of injector 100, catheter adapter 301 and gun 400, in accordance with an exemplary embodiment of the invention.

### Exemplary Variants

In an exemplary embodiment of the invention, the drug delivery system is configured to apply multiple shots using a single cocking of spring 417. In one example, spring 417 re-cocks each time it fires. In another example, rod 420 is used as a motion limiter (e.g., includes an axially translatable nut) and for each firing is adjusted to allow additional motion. Optionally, pin 410 is configured to selectively engage one of a plurality of aperture sin piston 408. In another example, the leading side of piston 408 is stepped around its circumference and pin 410 rather than being moved away from piston 410 is rotated around the circumference of piston 410, to a position which allows some motion of piston 408.

In some cases, re-cocking also includes advancing the main medicament piston in the injector, to provide washing of the system, repressurizing of the medicament (if needed) and/or ensure good contact between the pulse piston and the medicament. This may be provided, for example, manually or automatically. Optionally, a larger medicament volume is provided, for example, double or triple and optionally including provision for wastage, such as 50% or 70% wastage.

In an exemplary embodiment of the invention, additional medicament is provided into injector 100 between firings, if needed, for example, through a one way valve in body 102 (not shown).

While a mechanical device is shown, in an alternative embodiment of the invention, a hydraulic system is used, with, for example, a hydraulic accumulator to provide the pulse to move pistons 408 and/or 108. Optionally or alternatively, a pneumatic spring is used and optionally replaced between firings. Optionally or alternatively, an explosive charge, optionally electrically activated, is used to move piston 408.

In an alternative embodiment of the invention, the system is electrically or electronically controlled, for example, causing release of piston 408 and/or cocking of the piston under electrical control. Such control may be automatic, or may be manual (e.g., buttons). Optionally, sensors are provided for supporting automatic operation and/or safety, for example, medicament and/or inner balloon pressure sensors.

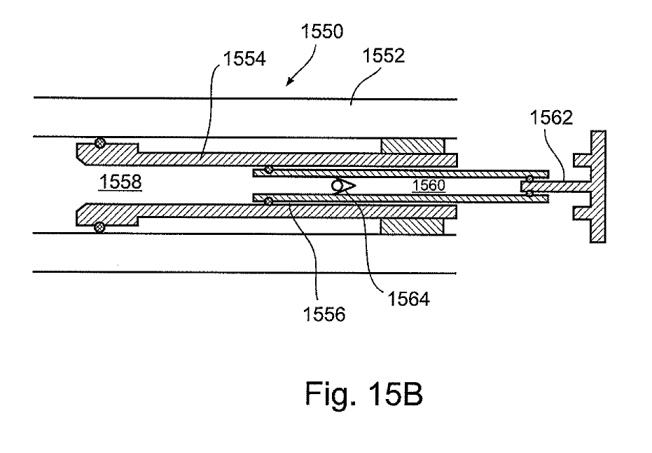

FIGS. 15A and 15B show exemplary injector designs that store medicament therein in a multiple part (e.g., two part) form, which may allow a longer shelf life.

FIG. 15A shows an injector 1500, in which a body 1502 is sealed using a seal 1504, optionally removed before use or torn by inserting into the catheter adapted. A first piston 1510 defines a hollow 1506 for a first medicinal component. In side of piston 1510, a second piston 1512 (e.g., corresponding to piston 108) defines a second hollow 1508, optionally sealed by a membrane 1514, containing a second medicament component. Advancing of piston 1512, can tear membrane 1514 and allow the medicament components to mix. Multiple hollows with separating membranes may be provided inside piston 1510 and the membranes all torn by advancing of piston 1512.

FIG. 15B shows an alternative multi-compartment injector 1550, in which a first medicament component is stored in a hollow 1558 defined by an outer piston 1554 and a body 1552, and a second medicament component is stored in a hollow 1560 stored within an inner piston 1556. A one way valve or trearable membrane 1564 is optionally breached by advancing of a plunger 1562 inside piston 1556. Optionally, when the plunger completes its forward movement, the plunger is coupled to piston 1556 and serves the function of piston 108, above.

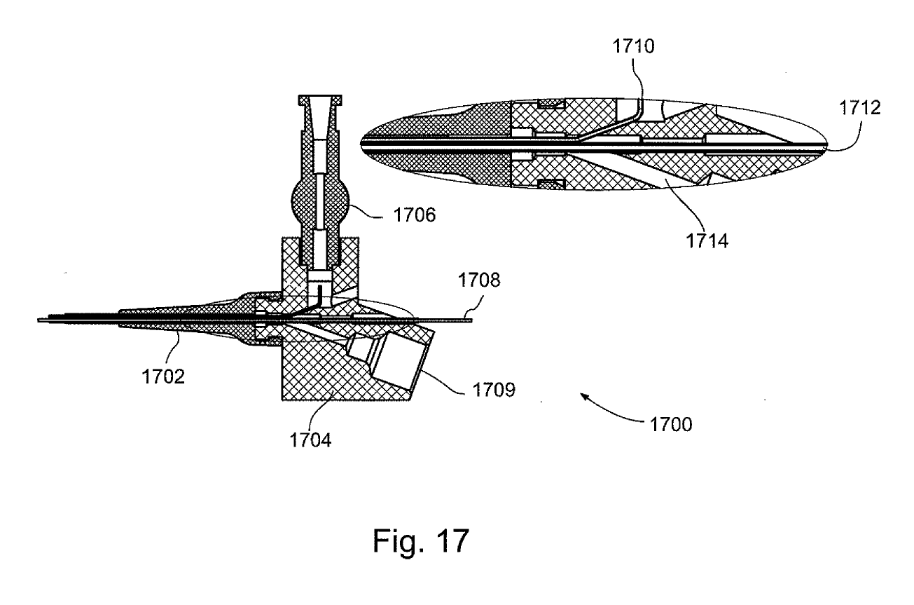

FIG. 17 shows an alternative catheter adapter 1700, which may be useful for peripheral vessel usage and/or with a guide wire. Adapter 1700 includes a body 1604, to which a pressure source (e.g., a PTCA pump) can be coupled to an inner balloon lumen 1710 (e.g., via adapter 1706, which may include a valve or a narrowing as described herein). A guide wire 1708 is optionally provided via a straight channel 1712, to reduce possible kinking thereof and/or other damage to the guidewire and/or difficulty in using. An outer balloon pressure and/or pulse pressure are optionally provided via a connector 1709 which couples to an outer balloon lumen 1714.

### Exemplary Kits

In an exemplary embodiment of the invention, the system for drug delivery is provided in kit for, for example, including all needed element or including disposable elements.

In one example, a kit includes a pulse gun, medicament, a PTCA pump, a catheter (or other delivery tip and an injector. Optionally, the medicament is provided in the injector, as one component or as several separated components. Optionally, the gun and/or PTCA pump are omitted.

Optionally, the kit includes a stent, which may be carried on the balloon valve or be provided with a separate catheter.

Optionally or alternatively, the kit includes also, or only a plurality of balloon valves (e.g., with catheters of optionally varying diameter) and/or plurality of medicaments and/or a plurality of injectors. Optionally, the various components are matched (e.g., using a table) with respect to a desired effect on the body. Optionally, different injectors have different volumes and/or different per-pulse injection amounts.

Optionally or alternatively, the kit includes one or more spacers or other elements that modify the pulse size and/or volume, for example, as described herein.

In an exemplary embodiment of the invention, a kit includes instructions for use, for example, in printed form, in electronic form or as a link to a remote location. Optionally, such instructions include knob settings for the elements, such as the injector.

### Materials and Reusability

In embodiments of the invention, the catheters are constructed of a flexible material. Examples of flexible materials suitable for use in embodiments of the invention include, but are not limited to, silicone, nylon and polyurethane. Optionally, parts of the devices are constructed of a rigid material, for example PVC (polyvinylchloride) or milled steel.

One consideration in material selection is a degree of disposability of the device (e.g., injector 100, catheter adapter 301 and/or gun 400 and/or tips and/or catheters). In embodiments of the invention, the device is constructed to be durable through a single use, and then discarded. According to various embodiments of the invention, the single use can comprise a number of hours (e.g. 2, 4, 8, 12 or 24 hours or lesser or grater or intermediate numbers of hours) or a number of days (e.g. 1, 2, 4, 7, 14 or 21 days or lesser or greater or intermediate numbers of days). One production consideration is that, a cost of materials may increase as multiple uses are allowed (e.g., requiring multiple pulses to not damage the device). In embodiments of the invention, a single use device is provided as an individual sterile sealed unit (e.g. in a bag), optionally contained within an applicator.

In other embodiments of the invention, the device is adapted for re-use. Optionally, the device is constructed to withstand repeated sterilization (e.g. by steam pressure, formaldehyde gas or UV irradiation or dry heat) and/or washing (e.g. with detergents and/or solvents).

In an exemplary embodiment of the invention, various parts of the device are adapted to ensure compliance with single use instructions. In one example, once attached, parts cannot be detached without breaking. Optionally or alternatively, the gun cannot be re-cocked. Optionally or alternatively, the pressure valves are one time and/or do not zero once used. Optionally or alternatively, the trigger cannot be re-pressed.

### General

It is expected that during the life of a patent maturing from this application many relevant urinary incontinence devices will be developed and the scope of the term urinary incontinence device is intended to include all such new technologies a priori. As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A medicament injector, comprising:
(a) a hollow body having an outlet;
(b) a first piston configured to increase a pressure inside said hollow body; and
(c) a second piston configured to increase transmit a pressure pulse into said hollow body.

2. An injector according to claim 1, wherein said second piston travels within said first piston.

3. An injector according to claim 1, including a manual handle for advancing said first piston.

4. An injector according to claim 1, comprising a pressure release pathway defined between said pistons and coupled to said hollow of said hollow body during only part of a relative position of said two pistons.

5. An injector according to claim 1, prefilled with at least one medicament component.

6. An injector according to claim 5, prefilled with at least two medicament components, each in a separate chamber of said injector.

7. An injector according to claim 3, comprising a rotational mechanism which advances said first piston.

8. A medicament delivery system comprising an injector according to claim 1, a pressure pulse source coupled to said second piston and a medicament delivery tip coupled to said outlet.

9. A method of delivering a medicament, comprising:
(a) pre-pressuring a delivery tube using a medicament injector; and
(b) applying a pressure pulse to said delivery tube using said injector to pass on a pulse.

10. A method according to claim 9, comprising attaching a pulse source to said injector after navigating said delivery tube to a treatment area.

11. A method of assembling a medicament delivery system, comprising:
providing a pulse gun;
selecting from a plurality of application tubes, suitable for different tissue types, an application tube;
attaching said application tube to said gun.

12. A method according to claim 11, comprising selecting a medicament source from a plurality of available sources.

13. A method according to claim 11, comprising adjusting a pulse setting of said system according to said selection.

14-22. (canceled)
